SEQUENCE LISTING (1 sequence)

SEQ ID NO: 1 - protein, 655 aa, Toxoplasma gondii
MAMQLPADDETEGQSEDNRNFSAIWEVPAPHTESLDGQLGVLLNNGSLTDREEVTEFQPLTSQTGSAGNGNDSQMDVASEELDSLVKNDQGQVTSTMAETSTRLEATEMSLPSKSESNAVDEATATATPEGSLSASLPHARKPTDEEEYYLGKVGHEEQLKRDTDSEHSEMNPEGTGVLHSRAEEKDAAHADSEGDRAGGADTVAEMKGEQHHEGLLGEGGEGMLNKEMHIRKEVDSLKGTSAKSGPEGEAQKQQYNGEHDEQVVMSPALLESTNTVTPTSEQTMADIADMDELEEIHETMMTANSPSIIVGSEPSALEPAEVSERAYEPSMGPLDAASLGTSEVAPHSVLSPAIVDTSSSDQQGTVSPSSGLSLLPGNVEGANVTQGMLLSGNSGDQITDVPSSTESCHIQTVHPSHSHNGLMEPASHQDLHLMHESRGPHEMERTVLDAESGILPGSQLHDESGPLLGVLPAASSSHSGHGFVHEGSHIFTEPADIHGLPDTGVHESIHSTFPMSAAHEAPLGVGGHGVISSPEPTQTNLLPPMATAVQAIQSPFLAHETSPAFSTPMHVSSSTGELVTVPGALAFVQSEEAAATRGGKKKGGNKNKKSPPAVVYESKPGPMVTEHKPTVHIAEPILVELQEKPKKRSRRAPI

(19) United States
(12) Patent Application Publication
(10) Pub. No.: US 2012/0189581 A1
(43) Pub. Date: Jul. 26, 2012

(54) USE OF TOXOPLASMA AND DERIVED COMPOSITIONS TO PREVENT OR TREAT MICROBIAL INFECTIONS

(76) Inventors: Stacey L. Schultz-Cherry, Germantown, TN (US); Laura Knoll, Madison, WI (US); Lori Neal, Madison, WI (US); Kevin O'Brien, Memphis, TN (US)

(21) Appl. No.: 13/386,784

(22) PCT Filed: Jul. 23, 2010
(86) PCT No.: PCT/US10/43115

§ 371 Date: Apr. 10, 2012

Related U.S. Application Data
(60) Provisional application No. 61/271,704, filed on Jul. 24, 2009.

(51) Int. Cl. A61K 35/68; A61K 38/44; A61P 31/16; A61P 31/10; A61P 31/04; A61P 31/12; C12N 1/10; A61P 31/00

(52) U.S. Cl. 424/93.1; 435/258.1; 424/780; 424/94.4

(57) ABSTRACT

The invention provides a composition comprising an amount of a live attenuated or avirulent Toxoplasma gondii strain or a soluble extract thereof or a soluble extract of a wild-type T. gondii strain effective to inhibit or prevent heterologous microbial pathogen infection or replication, or one or more symptoms or manifestations of the microbial pathogen infection or replication, and methods of making or using the compositions.

### CROSS-REFERENCE TO RELATED APPLICATIONS

This application claims the benefit of the filing date of U.S. application Ser. No. 61/271,704, filed on Jul. 24, 2009, the disclosure of which is incorporated by reference herein

### STATEMENT OF GOVERNMENT RIGHTS

The invention was made with support from the Government of the United States of America (grants AI059049 and AI054603 from the National Institutes of Health). The Government has certain rights in the invention.

### BACKGROUND

Outbreaks of influenza A virus continue to cause widespread morbidity and mortality worldwide. Annually, more than 3.5 million people may die of influenza infection or influenza-related complications worldwide. Furthermore, to date, over 300 humans have been infected with the highly pathogenic avian H5N1 influenza viruses, i.e., bird flu, with a 70% mortality rate, leading to continued concern that these viruses may cause the next influenza pandemic.

The primary options available to combat influenza virus are vaccination and antiviral drug therapies. While the establishment of comprehensive vaccination policies has been an effective measure to limit influenza morbidity, the frequent genetic drifting of the virus requires yearly reformulation of the vaccine, potentially leading to a mismatch between the viral strain present in the vaccine and that circulating. Further, since the identity of the virus in the vaccine is largely based on an estimate of the viruses from the previous year's influenza season, there is no guarantee of vaccine efficacy. Additionally, influenza vaccines comprising inactivated influenza virus must typically be given in high concentrations in order to bring about a significant increase of antibodies, and the administration of inactivated influenza virus or antigen in convenient commercial doses, free of side effects, does not always produce a satisfactory immune response, particularly when administered nasally or orally. Vaccines and effective therapies against H5N1 viruses are also limited. Thus, antiviral therapies against influenza virus are important tools to limit both disease severity as well as transmission.

Currently, there are two classes of influenza antivirals approved for widespread distribution, including adamantane derivatives (e.g., amantadine and rimantadine), and neuraminidase inhibitors. The adamantines target the viral M2 protein, and prevent the virus from uncoating and releasing its genetic material into the cell. In contrast, the neuraminidase inhibitors (NAIs), block the enzymatic activity of the neuraminidase (NA) surface protein, and halt viral egress. Unfortunately, there are increasing reports of emerging viruses resistant to both classes of antivirals. Due to large scale resistance, the Centers for Disease Control and Prevention and others have recommended against the use of the adamantanes for treatment or prophylaxis of influenza viruses. Additionally, neuraminidase inhibitors, such as oseltamivir, are generally efficacy-limited to administration within 48 hours of infection, or close to the onset of symptoms.

With a constant pandemic threat and an increasing population susceptible to severe infection with seasonal influenza, there is an urgent need to identify and characterize new antiviral drugs for both treatment and control of influenza that are effective, easy to administer, and cost effective.

### SUMMARY OF THE INVENTION

The present invention relates to immunomodulatory compositions and methods which employ live attenuated, for example, avirulent, e.g., Toxoplasma, e.g., Toxoplasma gondii including tachyzoites, bradyzoites and/or oocysts, or soluble extracts thereof, e.g., recombinant attenuated or avirulent, e.g., Toxoplasma which may express one or more heterologous gene products or soluble extracts thereof, soluble extracts of wild-type Toxoplasma, inactivated Toxoplasma, e.g., inactivated via chemical or heat treatment, one or more isolated T. gondii protein(s), or recombinant cells expressing one or more Toxoplasma proteins, as well as isolated native or recombinant lipoxygenase from Toxoplasma or other sources, e.g., plant lipoxygenase. In one embodiment, attenuated strains useful in the composition and methods of the invention are those that are attenuated during acute infection, produce fewer bradyzoite cysts in the brain over time, are not persistent as bradyzoites, e.g., strains including N28E2 and 41E2, have a reduced ability or an inability to convert from bradyzoites to tachyzoites, or an attenuated phenotype in immunocompromised mammals, e.g., strains including 9×G4 and 95C5, or any combination thereof. As described herein below, compositions including those having inactivated and/or avirulent or attenuated, e.g., T. gondii or a soluble antigen extract of T. gondii tachyzoites, were found to inhibit the effects of various microbial infections and reduce the mortality rate and morbidity in mammals administered an otherwise lethal dose of infectious microbes.

The invention provides compositions and methods for beneficially modulating an immune response to a variety of different microbial pathogen infections, e.g., viral, bacterial, fungal or parasitic infections, in animals including avians and mammals. Thus, the compositions of the invention, for example, a single dose thereof, are broad spectrum immunotherapeutics and, as disclosed herein, provide for prophylactic and/or therapeutic activity against a variety of diverse microbes. In one embodiment, the method includes administering to a mammal having or suspected of having a microbial pathogen infection, e.g., a mammal exposed to a microbial pathogen, a composition comprising an effective amount of a live avirulent or attenuated e.g., Toxoplasma gondii strain or a soluble extract thereof, a soluble extract of wild-type Toxoplasma gondii strain, a recombinant Toxoplasma gondii strain that expresses one or more immunogens of the pathogen or a soluble extract thereof, one or more isolated proteins, e.g., isolated lipoxygenase such as isolated plant or Toxoplasma lipoxygenase (see www.toxodb.org; Accession Nos. TGME-115970 (SEQ ID NO:1), TGME49-063410, or TGME49-067410, the disclosures of which are incorporated by reference herein), or recombinant cells expressing one or more Toxoplasma proteins. In one embodiment, the method employs extracts of T. gondii, e.g., wild-type or recombinant attenuated or avirulent strains, or combinations thereof.

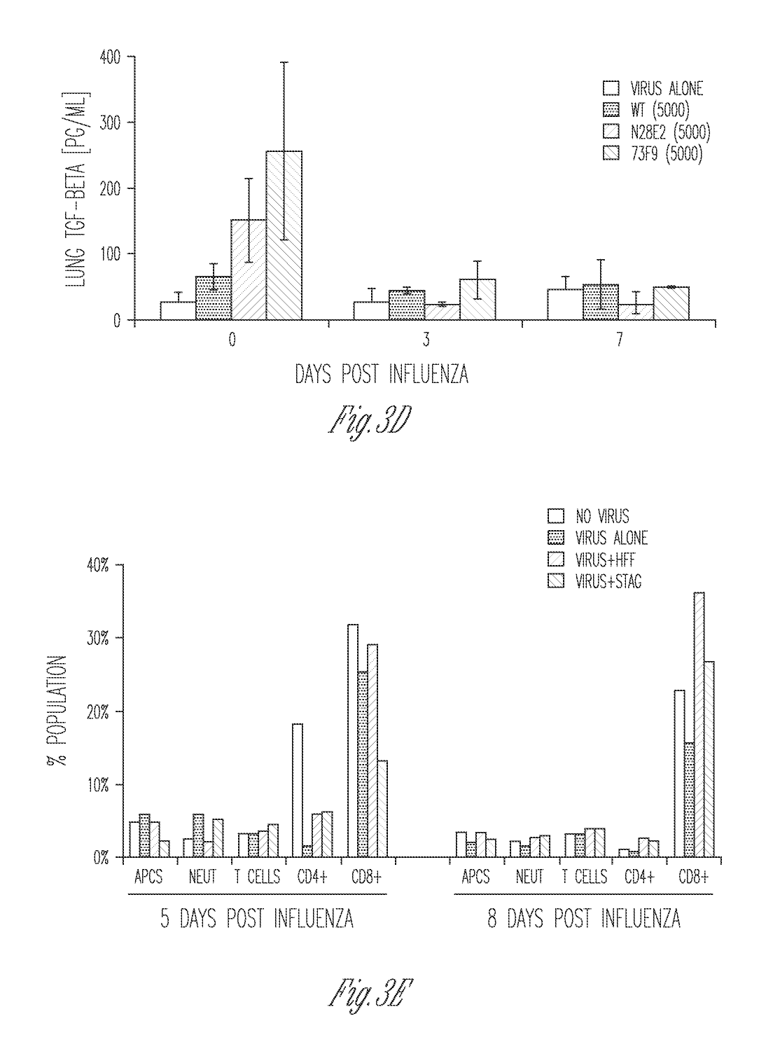

In one embodiment, the use of a composition of the invention having one or more gene products of Toxoplasma, e.g., soluble extracts of wild-type or avirulent T. gondii (for example, a soluble antigen extract of T. gondii tachyzoites, “STAg”), or corresponding gene products of other organisms, e.g., plant lipoxygenase, may prevent, inhibit or treat heterologous (non-Toxoplasma) microbial infection, e.g., infection by a virus, bacterium, fungus or parasite, of an animal, such as an avian or a mammal including human and non-human mammals. In one embodiment, the composition comprises about 100 μg/mL to about 2000 μg/mL, e.g., about 150 to 300 μg/mL, or about 10 mg to about 1000 mg, e.g., about 20 mg to about 500 mg, or 100 to 1000 μg, e.g., 200 μg, of extracted Toxoplasma gondii protein per dose. For example, after infection of C57B/6Ncr mice with wild-type or attenuated T. gondii strains, allowing for establishment of early (1 month) or late (4 month) chronic T. gondii infection, mice were intranasally infected with A/Hong Kong/483/1997 (HK/483) and monitored for disease. Alternatively, mice were first inoculated with HK/483 virus followed by administration of a T. gondii extract 48 hours post-influenza infection. At different times post-influenza infection, tissues were isolated; viral titers, histopathology, and cytokine levels were examined. Inoculation with wild-type and attenuated strains of T. gondii 1 month prior (early chronic stage) to HK/483 infection protected about 70 to 100% of the virus infected mice from mortality as compared to 100% mortality in the untreated group. T. gondii-mediated protection was dose- and life-cycle stage-dependent depending on the parasite strain. In addition, influenza infection apparently “cured” the mice of T. gondii, increasing the benefits to the host (see FIG. 5). Further, a STAg preparation enhanced survival, lowered viral titers, and reduced clinical disease when administered 48 hours after H5N1 influenza infection of mice, the latest point at which currently available influenza drugs are effective. In particular, 80% of mice treated with a soluble extract of T. gondii 48 hours after lethal H5N1 influenza virus infection survive and have significantly lowered viral titers. More importantly, survivors were protected from subsequent re-infection; suggesting that potent memory responses were generated. Of great importance, only 1 dose of extract was required for increased survival and decreased viral titers.

In one embodiment, the administration of a composition of the invention to avians or mammals provides for enhanced survival after exposure to influenza virus, e.g., survival rates of at least 35% or greater, for instance, survival rates of 50%, 60%, 70%, 75%, 80%, 85%, 90% or greater, relative to survival rates in the absence of the administration of that composition or any other prophylactic or therapeutic agent. The compositions of the invention are also useful prophylactically or therapeutically against seasonal flu and other viral infections.

In one embodiment, a composition of the invention may be employed to inhibit a microbial infection. For instance, pretreatment of mice with isolated soybean lipoxygenase or STAg preparations reduced the number of colony forming units in liver and spleen of mice subsequently exposed to Listeria.

In one embodiment, a composition of the invention may be employed to treat a microbial infection. As described hereinbelow, mice exposed to lethal infectious influenza virus survive if subsequently treated with STAg from wild-type or attenuated T. gondii. The T. gondii treatment was effective for at least two days post-influenza infection, and likely several days more, which suggests that such T. gondii products may be a more effective treatment than the current influenza antiviral drugs.

Accordingly, in one embodiment, the invention provides compositions and methods for preventing and limiting influenza infection that may be more persistent and easier to develop than current methods, given that new vaccines would not need to be developed each season. Moreover, the use of the compositions of the invention may be less likely to result in resistant influenza strains than current antiviral approaches. Further, the composition may comprise combinations of the aforementioned compositions with influenza virus immunogens (antigens), e.g., HA and NA, for instance, recombinantly expressed in T. gondii.

As also described hereinbelow, STAg was an effective treatment for L. monocytogenes infections when administered 24 hours after bacterial infection. Further, proteinase K treatment of STAg ablated its protective effects for L. monocytogenes, indicating that the active component in STAg is likely a protein. Moreover, STAg was effective as a treatment for influenza and L. monocytogenes regardless of the route of injection (equally effective by intraperitoneal (ip) or intravenous (iv) injection). In addition, pretreatment of mice with STAg or isolated lipoxygenase (e.g., one from a source other than a STAg preparation) reduced Listeria colonies in liver and spleen of infected mice. In one embodiment, the administration of a composition of the invention to avians or mammals provides for decreased bacterial colonization after exposure to bacterial infection, e.g., at least a 1 or 2 log drop in colony forming units relative to colony forming units in the absence of the administration of that composition or any other prophylactic or therapeutic agent.

Also, most of the C57BL/6 mice that were infected with Plasmodium berghei, then treated with STAg at 48 and 96 hours post infection were asymptomatic up to 20 days post infection when the experiment was ended. The data highlight the broad spectrum antimicrobial activity of STAg, and proteins in STAg preparations, and its diverse possible uses. For example, STAg activity against P. berghei is indicative that STAg is likely effective against other category B coccidian parasites such as Cryptosporidium parvum and Cyclospora cayatanensis.

As broad spectrum immunomodulators, the compositions of the invention are useful for rapid prophylaxis or treatment with only minimal available diagnostic data. Because the induction of an adaptive immune response is not necessary for protection by the compositions of the invention, as disclosed herein, those compositions are likely to be effective in T cell deficient mammals, e.g., humans with HIV, autoimmune disorders, organ transplant recipients, and calcineurin inhibitor recipients, and the administration of those compositions is less likely to result in the development of drug resistance by the infecting microbe. Further, augmenting the innate immune system in immunocompetent or immunocompromised avians and mammals in combination with traditional antimicrobials may decrease the pressure of microbes to develop resistance.

In one embodiment, the method includes administering to a mammal having or suspected of having a microbial pathogen infection, a composition comprising an effective amount of a live avirulent or attenuated Toxoplasma gondii strain or a soluble extract thereof, a soluble extract of wild-type Toxoplasma gondii strain, inactivated T. gondii, a recombinant Toxoplasma gondii strain that expresses one or more immunogens of the pathogen or a soluble extract thereof, or one or more isolated proteins, e.g., isolated lipoxygenase. In one embodiment, the method employs soluble extracts of T. gondii, e.g., from wild-type or recombinant attenuated strains, e.g., 73F9 and N28E2, or combinations thereof. In one embodiment, the method employs inactivated T. gondii. In one embodiment, the composition further comprises a pharmaceutically acceptable carrier. In one embodiment, the composition is administered orally, for instance, in a formulation suitable to deliver isolated protein(s). In another embodiment, the composition is administered through various other acceptable delivery routes, for example, through parenteral injection, intranasally, or via an intra-muscular injection. In one embodiment, the composition is administered to the mammal one or more times, at times including but not limited to 1 to 7 days, 1 to 3 weeks or about 1, 2, 3, 4 or more, e.g., up to 6, months, before the mammal is exposed to the pathogen. In one embodiment, the composition is administered to the mammal one or more times after exposure to the pathogen, e.g., at 1 hour, 6 hours, 12 hours, 1 day, 2 days, 4 days or more, e.g., up to about 2 weeks, after exposure. In one embodiment, the composition is administered to the mammal when the mammal is symptomatic. In one embodiment, the administration of a composition of the invention results in an increase in CD8⁺ T cells in the mammal. In one embodiment, the administration of a composition of the invention results in an increase in neutrophils in the mammal.

In one embodiment, the invention provides a method of immunizing a mammal against a microbial pathogen infection. The method includes identifying a mammal in need of immunization against the pathogen; and administering to the mammal a composition comprising an effective amount of a live avirulent Toxoplasma gondii strain or a soluble extract thereof, a soluble extract of wild-type Toxoplasma gondii strain, a recombinant Toxoplasma gondii strain that expresses one or more immunogens of the pathogen or a soluble extract thereof, or one or more isolated proteins, e.g., isolated lipoxygenase.

In one embodiment, a live avirulent Toxoplasma, e.g., Toxoplasma gondii, strain or soluble extracts thereof, a recombinant Toxoplasma strain or soluble extracts thereof, soluble extracts of a wild-type Toxoplasma strain, inactivated Toxoplasma strains, e.g., inactivated via chemical or heat treatment, isolated lipoxygenase, e.g., plant lipoxygenase, one or more isolated or recombinant T. gondii protein(s), e.g., isolated or recombinant lipoxygenase, or recombinant cells expressing one or more Toxoplasma proteins, is employed as an adjuvant to enhance the immune response to an immunogen (an adaptive immune response). Thus, in one embodiment, a composition of the invention may include one or more immunogens of a microbial pathogen (a non Toxoplasma microbe) and an amount of a live avirulent Toxoplasma strain, e.g., a Toxoplasma gondii strain, or soluble extracts thereof, a recombinant Toxoplasma strain or soluble extracts thereof, a soluble extract of wild-type Toxoplasma, inactivated Toxoplasma, e.g., inactivated via chemical or heat treatment, isolated lipoxygenase, e.g., plant lipoxygenase, one or more isolated or recombinant T. gondii protein(s), e.g., isolated or recombinant lipoxygenase, or recombinant cells expressing one or more Toxoplasma proteins, effective to enhance the immune response of an animal to the one or more immunogens, e.g., an inactivated or live avirulent microbe.

### BRIEF DESCRIPTION OF THE INVENTION

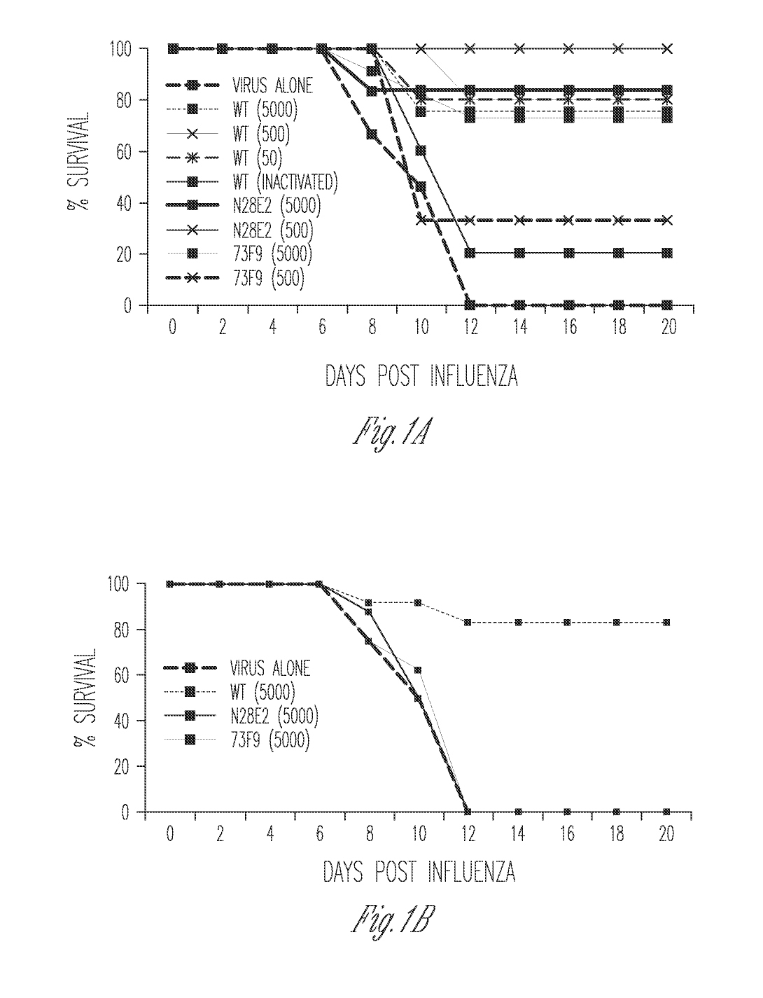

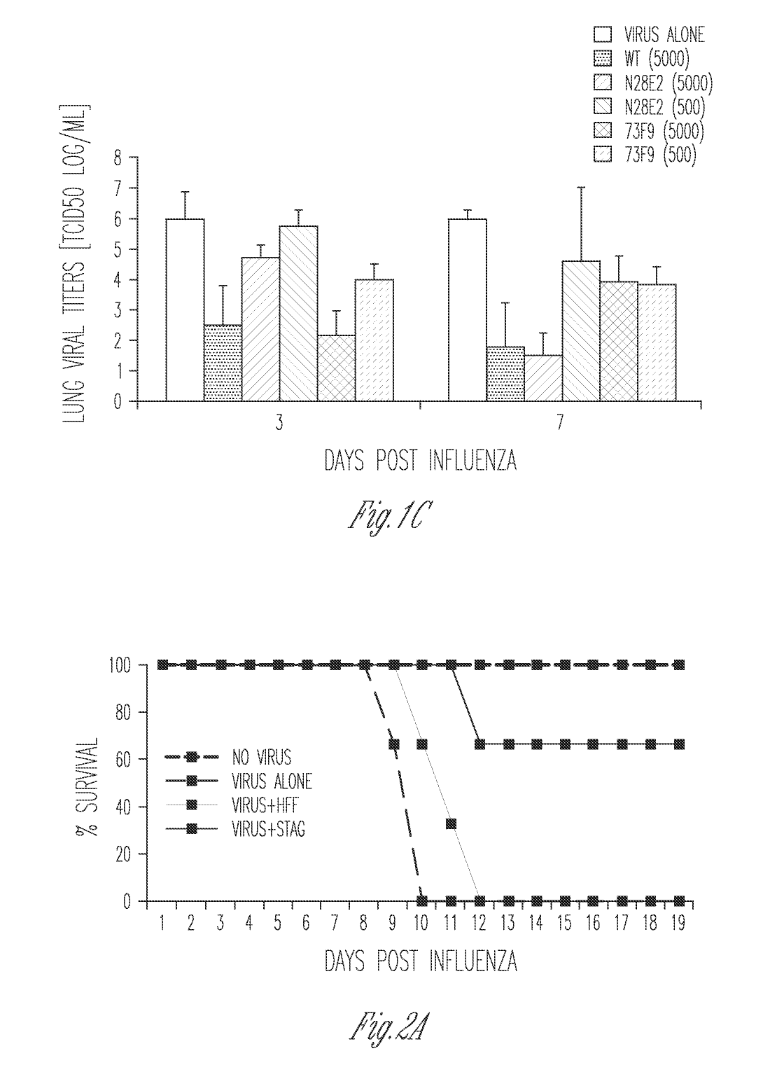

FIG. 1A. Chronic infection of T. gondii protects against H5N1 virus. Thirty days after infection with wild-type (WT) (red lines), N28E2 (blue lines), or 73F9 (green lines) strains of T. gondii (solid lines 5000, pixilated lines 500 and dashed line 50 parasites inoculated), mice were challenged with a lethal infection of HK/483 influenza virus. Age-matched controls kept in the same facility, but without prior infection of T. gondii (Virus alone, black line) all succumb to a lethal influenza challenge of by day 12 post infection (dpi). Similarly, mice infected with heat inactivated (HI) WT T. gondii were largely not protected (grey line).

FIG. 1B. Only chronic infection with WT T. gondii protects long-term against lethal influenza infection. Four months after infection with 5000 WT (red line), 5000 N28E2 (blue line), or 5000 73F9 (green line) parasites of T. gondii, mice were challenged with a lethal infection with 10⁴ viral particles of HK/483. Only mice with a chronic infection with WT parasites were protected. Age-matched controls kept in the same facility, but without prior infection of T. gondii (Virus alone, black line) all succumb to lethal influenza challenge or had to be euthanized by 12 dpi.

FIG. 1C. Viral titers in the lungs at 3 and 7 dpi were determined by TCID₅₀ analysis in Madin Darby canine kidney cells. Lungs were obtained from mice infected with T. gondii strains one month prior to influenza virus infection. By 3 days post influenza virus infection, titers were already reduced by 4-logs in mice previously infected with the highest concentrations of WT or 73F9 T. gondii (red and green solid bars respectively, p<0.009) and at least 1.5-2 logs in N28E2 infected mice (blue solid bar, p<0.01). At 7 dpi, viral titers in both the WT (p<0.01) and N28E2 infected mice (p<0.002) were only slightly above the limit of detection for the assay and some mice had completely cleared the viral infection. Viral titers were not significantly different in mice previously infected with 500 N28E2 parasites (pixilated blue bars) even though these mice were completely protected and even asymptomatic from influenza.

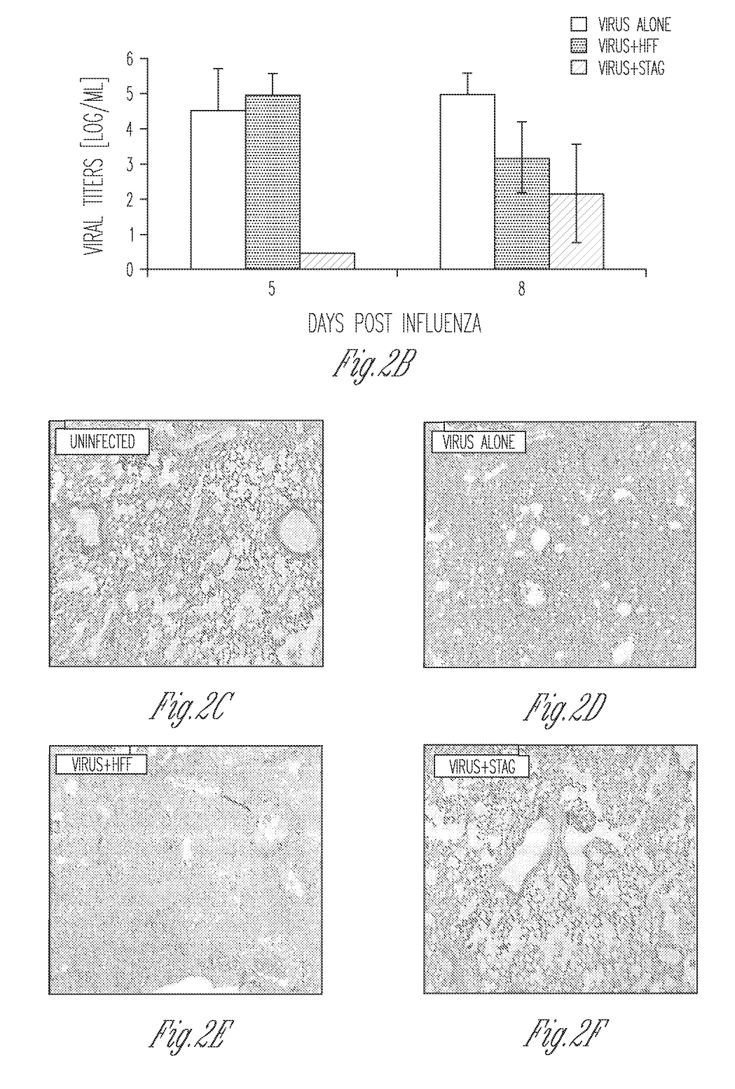

FIGS. 2A-B. Protection induced by treatment with STAg post-influenza infection A) Mice were either not infected with HK/483 influenza virus and treated at 2 and 4 dpi with PBS (No virus, green line) or infected with a lethal dose of HK/483 influenza virus and then treated at 2 and 4 dpi with either PBS (Virus alone, black line), human foreskin fibroblast (HFF) extract (Virus+HFF, blue line), or STAg (virus+STAg, red line). B) Viral titers in the lungs at 5 and 8 days post influenza infection as determined by TCID₅₀ analysis in Madin Darby canine kidney cells. By 5 dpi, viral titers in mice treated with STAg were below the limit of detection (red bars, p<0.001), compared to PBS treated mice (black bars). By 8 dpi, influenza titers in STAg treated mice were still significantly decreased as compared to PBS treated mice (p<0.003).

FIGS. 2C-F. Hematoxylin- and eosin (H&E)-stained lung tissue from unifected mice (panel C) or lung tissue (at day 5 post infection) from mice infected with avian influenza (HK/483) and administered a double dose (one dose at 48 hours and one dose at 96 hours post-infection with avian influenza) of PBS (panel D), human foreskin fibroblast cells (HFF) (panel E), a STAg preparation (panel F) prepared from the T. gondii strain N28E2. STAg treated mice show fewer inflammatory cells.

FIGS. 2G-H. Innate immune response of STAg drives a protective long-term immune response. G) Uninfected and STAg treated mice that survived the initial infection (from FIG. 2B) were challenged with ten times higher dose of HK/483 influenza virus (10⁵ particles) and monitored daily for weight loss and clinical signs of infection. All mice that had previously been infected with HK/483 influenza virus and then treated with STAg survived the ten times higher dose (STAg treated, red line), whereas the age-matched control mice that had not been previously infected with HK/483 influenza virus or treated with STAg succumb to infection by 10 dpi (no initial virus, blue line). H) The STAg treated mice were completely asymptomatic as can be seen by their steady weight (compare STAg treated red line with the no initial virus blue line).

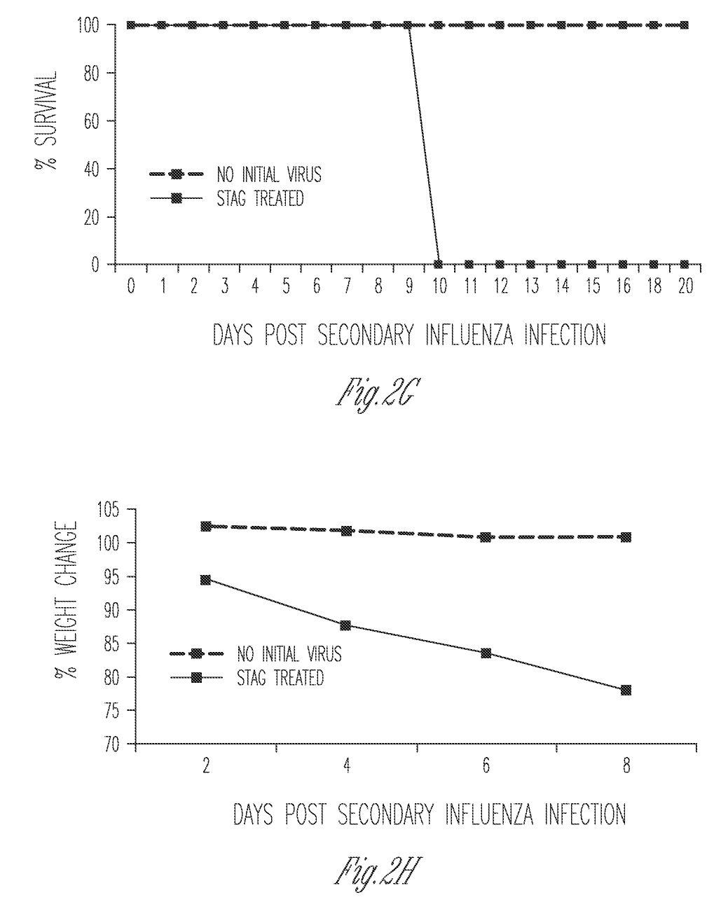

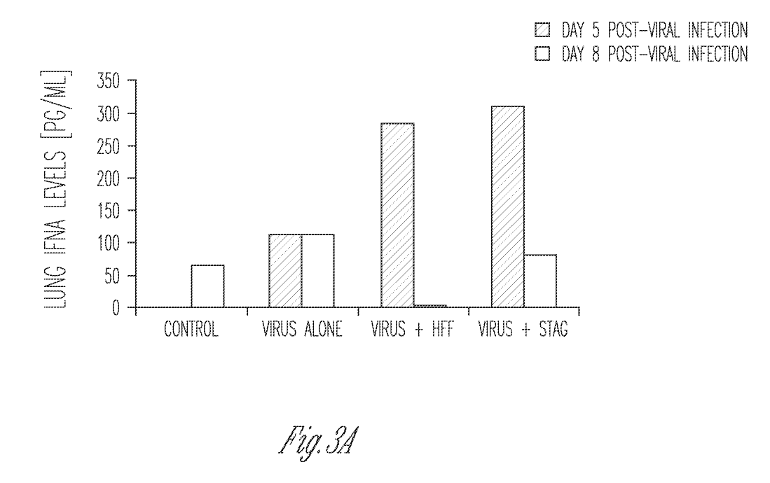

FIG. 3A shows the amount of IFN-alpha at days 5 and 8 post-influenza infection in the lungs of STAg-treated mice.

FIGS. 3B and E. STAg-treated mice do not have significant differences in their immune cell populations but do have higher interferon gamma. Mice were either not infected with HK/483 influenza virus and treated at 2 and 4 dpi with PBS (No virus, aqua) or infected with a lethal dose of HK/483 influenza virus and then treated at 2 and 4 dpi with either PBS (Virus alone, black), HFF extract (Virus+HFF, grey), or STAg (virus+STAg, red). At 5 and 8 dpi mice were sacrificed and ELISA (E) and flow cytometry (B) were performed on lung homogenates was performed as described in Tumpey et al. (2000). Each specific immune cell type was expressed as a percentage of the total number of viable lung cells. Neutrophils were counted as Ly6G/C⁺, and antigen presenting cells (APCs) were counted as F4/80⁺. T cells are first sorted as CD3⁺, then counted as CD4⁺ or CD8⁺. Thus CD4⁺ or CD8⁺ are expressed as a percentage of the total viable T cells. ELISA performed on lung homogenates showed that interferon gamma was significantly higher at 5 and 8 dpi in mice treated with STAg.

FIGS. 3C-D. Lung interferon gamma is increased 7 dpi in mice previously infected with T. gondii. Thirty days without prior infection of T. gondii (Virus alone, black) or after infection with WT (red), N28E2 (blue), or 73F9 (green) strains of T. gondii, mice were challenged with a lethal infection of HK/483 influenza virus. At 0, 3 and 7 dpi mice were sacrificed and ELISA performed on lung homogenates as described in Tumpey et al. (2000). Lung interferon gamma (panel C) was significantly higher at 7 dpi in mice that were previously infected with T. gondii. Lung TGF-beta (panel D) was consistently higher in T. gondii infected mice at 0 dpi, but low in all mouse lungs after influenza infection, indicating that highly pathogenic influenza may suppress TGF-beta production.

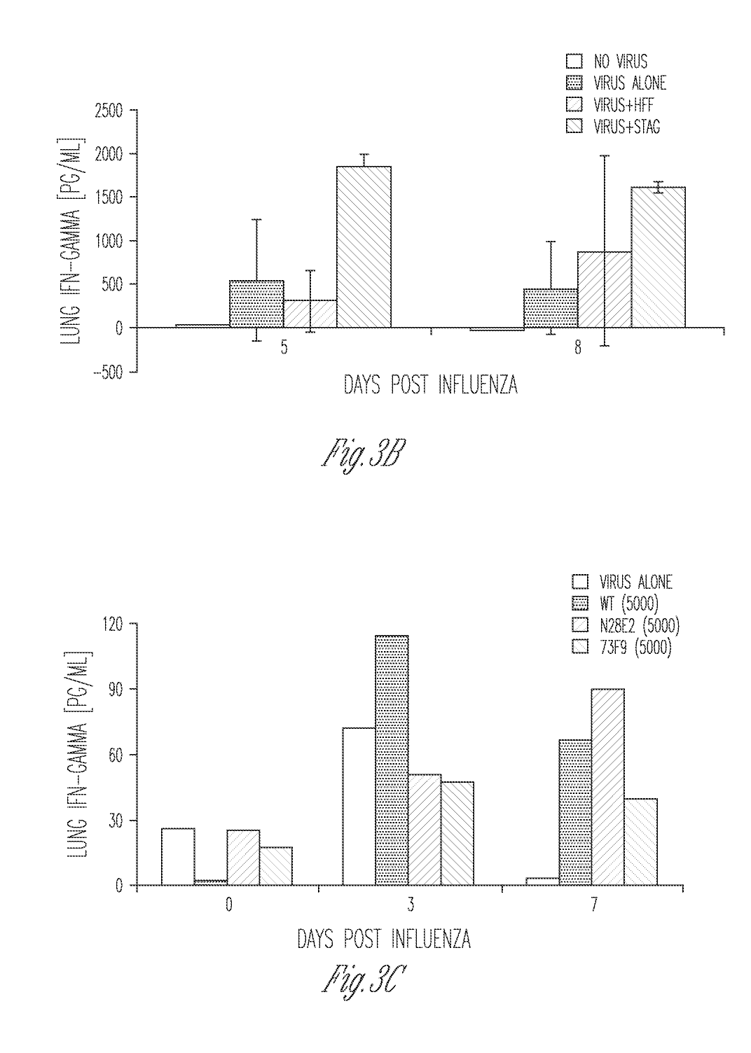

FIGS. 4A-B. STAg treatment still protective in Rag^{-/-} mice. A) RAG^{-/-} mice were either not infected with HK/483 influenza virus and treated at 2 and 4 dpi with STAg (Rag/STAg alone, blue line) or infected with a lethal dose of HK/483 influenza virus and then treated at 2 and 4 dpi with either PBS (Rag/virus alone, black line) or STAg (Rag/virus+STAg, red line). B) viral titers in the lungs at 5 and 8 days post influenza infection as determined by TCID₅₀ analysis in Madin Darby canine kidney cells. No significant differences were seen.

FIG. 5 illustrates the amount of T. gondii cysts found in the brains of mice pre-inoculated with wild-type T. gondii, the mutant T. gondii strains N28E2 or 73F9, or control mice (i.e., mice not pre-inoculated with T. gondii and not infected with influenza) at days 0, 3, 7, and 22 post infection with avian H5N1 influenza.

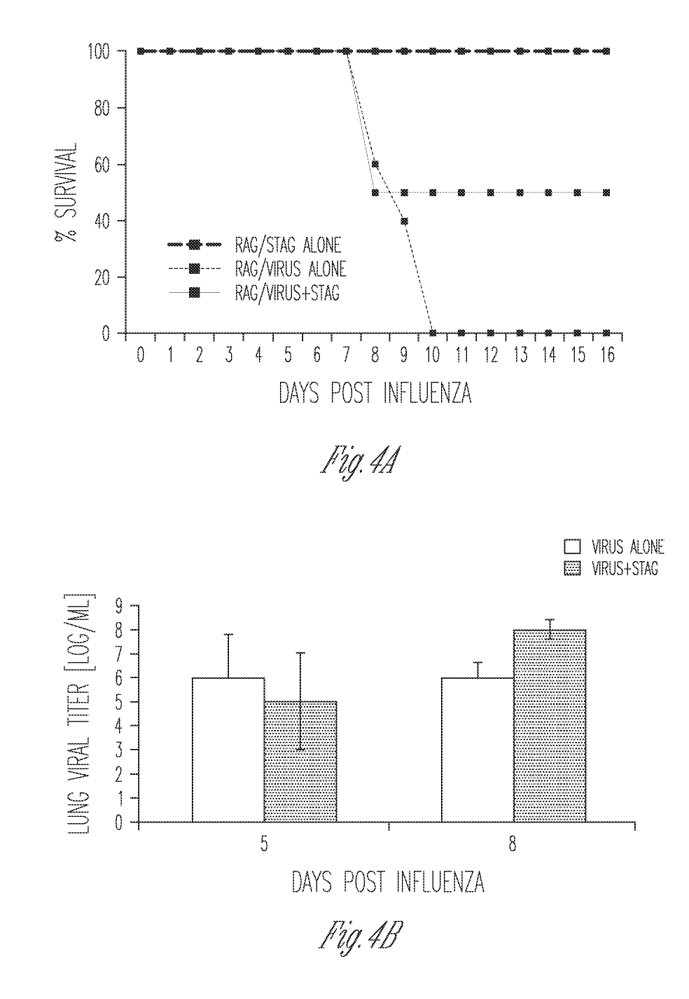

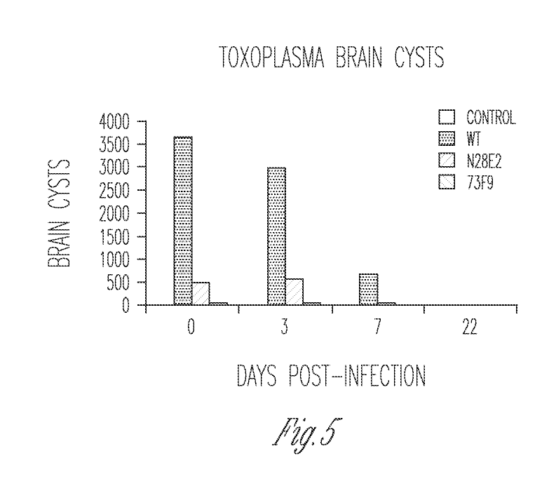

FIG. 6A shows day 0 flow cytometry results for mice pre-inoculated with either 5000 wild-type T. gondii or pre-inoculated with 5000 or 500 parasites from the mutant T. gondii strains N28E2 or 73F9 1 month prior to infection with avian influenza, as compared to an age-matched control group of mice that were not pre-inoculated with T. gondii and were not infected with influenza (control). The amount of neutrophils, antigen presenting cells (APC), CD4⁺ T cells, and CD8⁺ T cells are shown. The day 0 fold is compared to an age-matched control that did not receive T. gondii or influenza. The days 3 and 7 fold is compared to an age-matched control that did not receive T. gondii but were infected with influenza at day 0.

FIGS. 6B-C. Decreased viral titers correlate with increased neutrophils at 3 dpi and increased CD8⁺ T cells at 7 dpi. Thirty days after infection with WT (red), N28E2 (blue), or 73F9 (green) strains of T. gondii, mice were challenged with a lethal infection of HK/483 influenza virus. At 3 dpi (B) and 7 dpi (C) mice were sacrificed and flow cytometry on lung homogenates was performed as described in Tumpey et al. (2000). Shown is the percentage of immune cells in the lungs of T. gondii infected compared to age-matched controls kept in the same facility, but without prior infection of T. gondii (Virus alone, black). Each specific immune cell type was expressed as a percentage of the total number of viable lung cells. Neutrophils were counted as Ly6G/C⁺, and antigen presenting cells (APCs) were counted as F4/80⁺. T cells are first sorted as CD3⁺, then counted as CD4⁺ or CD8⁺. Thus CD4⁺ or CD8⁺ are expressed as a percentage of the total viable T cells.

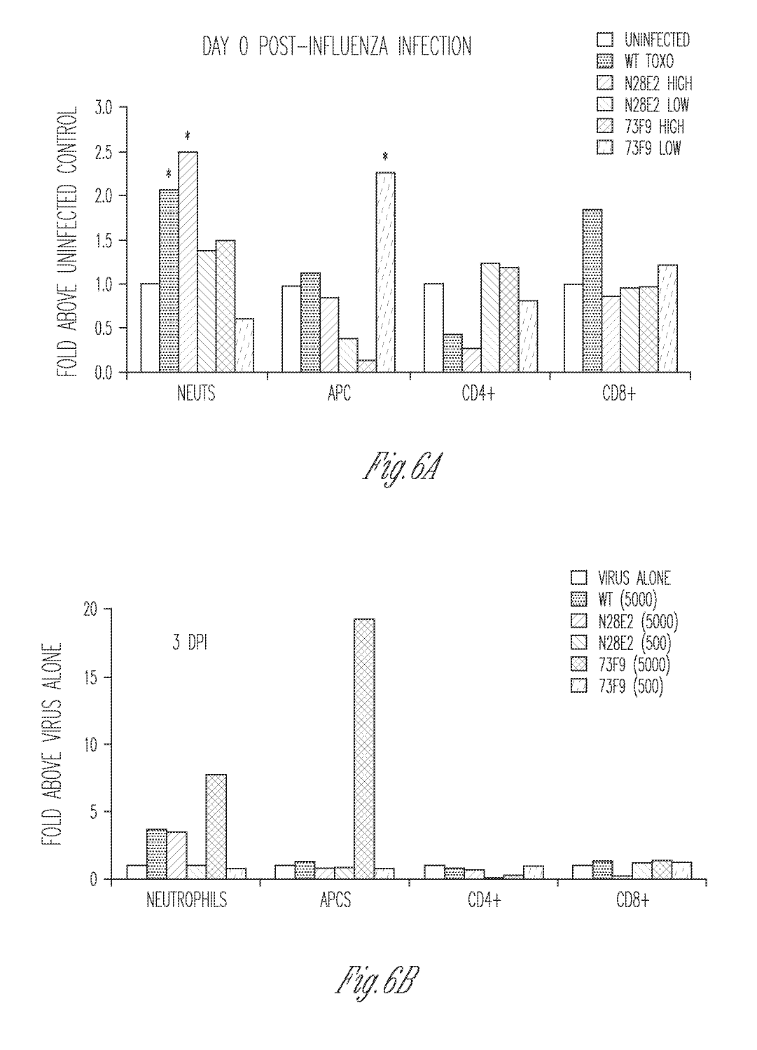

FIG. 7 depicts the amount of L. monocytogenes in the liver and spleen of mice infected with L. monocytogenes 24 hours after pretreatment with a STAg-containing preparation (prepared from the T. gondii strain N28E2), lipoxygenase, or PBS. C57BL/6 mice were pretreated 24 hours prior to L. monocytogenes infection with either 200 μg STAg (n=4), 10⁴ units soybean lipoxygenase (Sigma) (n=3), or PBS (n=4). Of the mice in each group, only 1 STAg treated mouse had any bacteria detectable and that was in the spleen. The limit of detection was about 2.5 CFU for liver and 3.2 CFU for spleen.

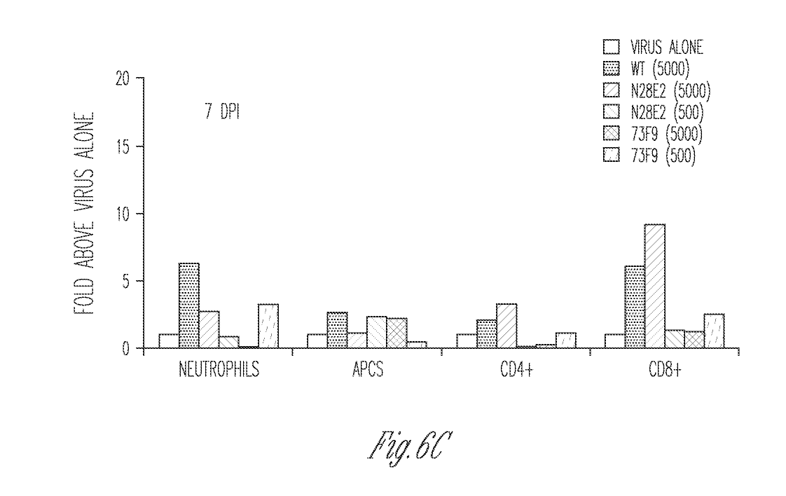

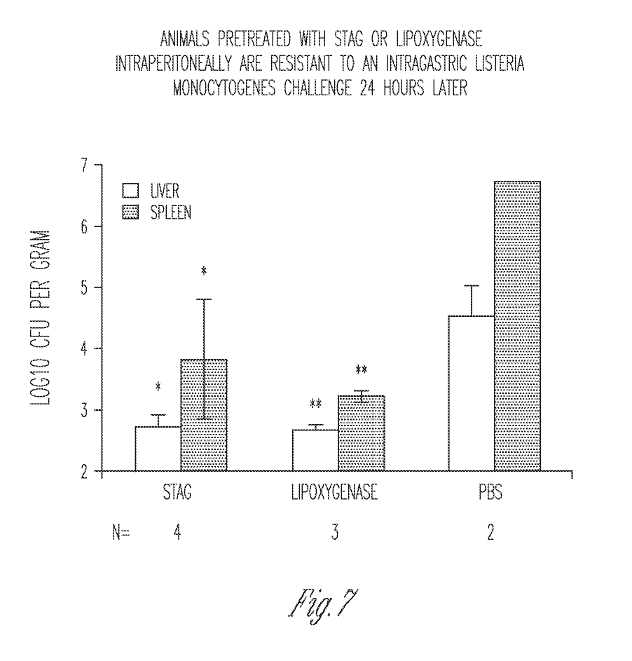

FIG. 8 depicts the amount of L. monocytogenes in the spleen of mice treated with a STAg-containing preparation, a STAg-containing preparation digested with proteinase K, or PBS 24 hours after infection with L. monocytogenes.

C57BL/6 mice (8 mice per group) were infected with L. monocytogenes, then treated 24 hours after infection with either 200 μg STAg, 200 μg of STAg digested with 100 μg/mL proteinase K, or PBS. Mice were sacrificed 3 days after L. monocytogenes infection, then their spleens ground, diluted, plated and the number of colony forming units (CFU) measured.

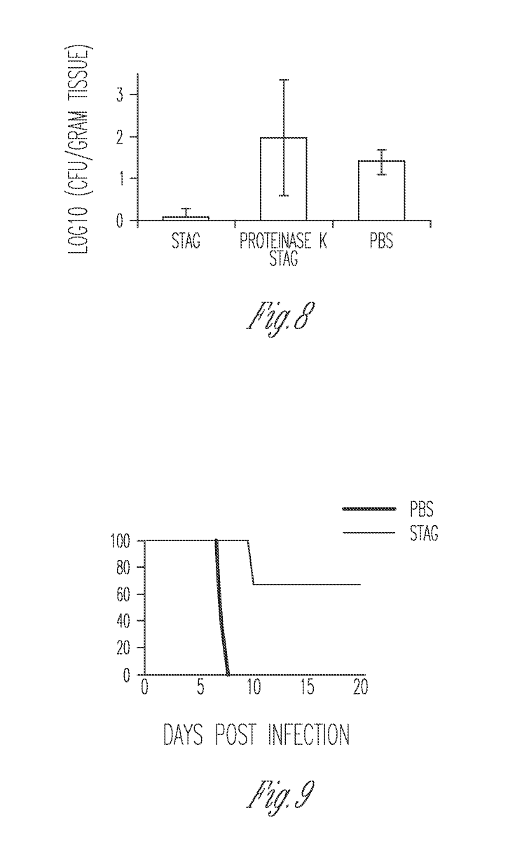

FIG. 9 shows the survival of mice infected with P. berghei and subsequently administered a double dose (one dose at 48 hours and one dose at 96 hours post-infection) of PBS, proteinase K alone, proteinase K treated STAg or a STAg preparation alone (prepared from the T. gondii strain N28E2). C57BL/6 mice were i.p. infected with 10⁶ red-blood cells parasitized with P. berghei (3 mice per group). At 48 and 96 hours post infection, mice were treated i.v. with about 200 μg of STAg. Activity was monitored every 12 hours starting at 5 days post-infection. Mice treated with PBS only were sacrificed at days 7 and 7.5 due to severe paralysis and convulsions. One of the three STAg treated mice was sacrificed at day 10 when it developed paralysis. The other two STAg treated mice were sacrificed at day 20 (end of the pilot), but were not symptomatic.

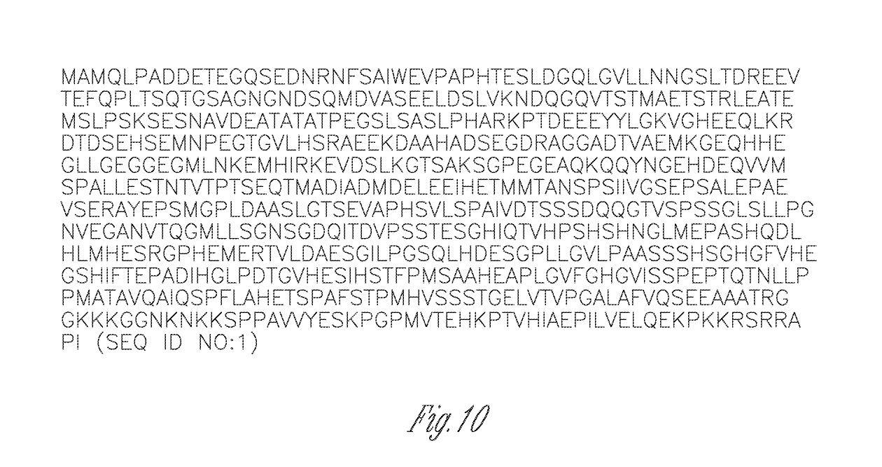

FIG. 10 sequence of putative Toxoplasma lipoxygenase (SEQ ID NO 1).

### DETAILED DESCRIPTION OF THE INVENTION

### Definitions

As used herein, the terms “isolated and/or purified” refer to in vitro preparation, isolation and/or purification of a microbial strain, cell or protein, so that it is not associated with and/or is substantially purified from in vitro or in vivo substances. An isolated strain or cell preparation of the invention is generally obtained by in vitro culture and propagation. A “recombinant” protein is one expressed using recombinant DNA techniques and a “recombinant” strain or cell is one which has been manipulated in vitro, e.g., using recombinant DNA techniques to introduce changes to the host genome For example, a “recombinant” strain or cell of the invention may be one which has been manipulated in vitro so as to contain an insertion and/or deletion of DNA in the genome, e.g., chromosome, of the strain or cell relative to the genome, e.g., chromosome, of the parent strain or cell from which the recombinant strain or cell was obtained (e.g., “wild-type” strain). In one embodiment, an insertion in the recombinant strain is stable, e.g., the insertion and its corresponding phenotype do not revert to wild-type after numerous passages. Included within the scope of the phrase “recombinant strain” is one which, through homologous recombination, includes a gene which contains a mutation that results in the inactivation of the protein in or reduced expression of the gene, e.g., results in a polypeptide having reduced or lacking biological activity or so that the polypeptide is not expressed, relative to a corresponding wild-type strain that does not include the recombined gene.

A “soluble extract” as used herein, includes soluble preparations of lysed cells or subcellular fractions thereof, that include components of any Toxoplasma strain, components such as one or more proteins, which may be prepared by any method. For instance, a soluble extract of Toxoplasma may be prepared by subjecting isolated Toxoplasma to sonication or a French press.

As used herein, an “attenuated” strain means a strain, the inoculation of which to a susceptible mammal, results in reduced (mild) symptoms or manifestations of Toxoplasma infection.

As used herein, an Aavirulent@ strain means a strain, the inoculation of which to a susceptible mammal, results in no clinical manifestations of Toxoplasma infection.

The term “isolated polypeptide” means a protein encoded by cDNA, recombinant RNA, a synthetic nucleic acid or any other nucleic acid, or some combination thereof, which by virtue of its origin the “isolated polypeptide” (1) is not associated with proteins found in nature, (2) is free of other proteins from the same source, (3) is expressed by a cell from a different species, or (4) does not occur in nature.

The term “operably linked” referred to herein refers to a juxtaposition wherein the components so described are in a relationship permitting them to function in their intended manner. A control sequenced “operably linked” to a coding sequence is ligated in such a way that expression of the coding sequence is achieved under conditions compatible with the control sequences.

The term “control sequence” referred to herein refers to polynucleotide sequences which are necessary to effect the expression of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is necessary for expression, and can also include additional components whose presence is advantageous, for example, leader sequences and fusion partner sequences.

The term “nucleic acid sequence” as referred to herein means a polymeric form of nucleotides of at least about 7 bases in length, either ribonucleotides or deoxynucleotides or a modified form of either type of nucleotide. The term includes single and double stranded forms of DNA.

As applied to polypeptides, the term “substantial identity” means that two peptide sequences, when optimally aligned, share at least 80 percent sequence identity, e.g., at least 90, 95 or 99, percent sequence identity. In one embodiment, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, glutamic-aspartic, and asparagine-glutamine. Whether a particular amino acid substitution results in a functional polypeptide can readily be determined by assaying the biological activity of the variant polypeptide by methods well known to the art.

### Exemplary Compositions and Methods of the Invention

The present disclosure generally relates to immunomodulatory compositions and methods for treating, inhibiting or preventing microbial infections in mammals. In particular, the present invention relates to immunomodulatory compositions and methods which employ live attenuated or avirulent Toxoplasma, e.g., Toxoplasma gondii, including tachyzoites, bradyzoites and/or oocysts, or soluble extracts thereof, e.g., recombinant attenuated Toxoplasma which optionally may express one or more heterologous gene products, or soluble extracts thereof, soluble extracts of wild-type Toxoplasma, inactivated Toxoplasma, e.g., inactivated via chemical or heat treatment, one or more isolated proteins, such as isolated native or recombinant lipoxygenase, from Toxoplasma, e.g., from T. gondii, Pseudomonas, for instance, P. aeruginosa, Brugia malayi, Trypanosome brucei, Schistosoma mansoni or noninfectious fungi, or other isolated Toxoplasma protein(s) from T. gondii, or other species, or recombinant cells expressing one or more Toxoplasma proteins. Methods for preparing and isolating recombinantly expressed proteins, e.g., proteins expressed as a His tag fusion in E. coli, are known to the art.

T. gondii is an intracellular parasite which is classified among the Coccidia. This parasite has relatively broad host range, infecting both mammals and birds. T. gondii is ubiquitous in nature and during the asexual cycle, occurring in any warm blooded animal, it exists in two forms: the tachyzoite and the bradyzoite. Tachyzoites, found during acute infection, are the invasive form capable of invading all nucleated mammalian cells. After the acute stage of infection, tissue cysts called bradyzoites are formed within host cells and persist within the host organism for the life of the host. Cysts are important in transmission of infection, especially in humans, as the ingestion of raw or undercooked meat can result in the ingestion of bradyzoites which can infect the individual resulting in an acute infection. Oocysts represent the end product of sexual reproduction which occurs only in the intestinal lining of the cat family from which they are excreted in the feces.

Surprisingly, T. gondii and T. gondii extracts (e.g., STAg), and homologs of T. gondii proteins, e.g., plant lipoxygenases, are effective at preventing, inhibiting or treating heterologous microbial infections, including viral infections such as influenza infection (including infection with pathogenic avian H5N1 viruses), bacterial infections such as infection with L. monocytogenes, and infections of various parasites such as Plasmodium (e.g., Plasmodium berghei and Plasmodium falciparum), which is known to cause cerebral malaria. In particular, administration of a culture of inactivated (chemically inactivated, e.g., with formalin, or heat inactivated) T. gondii, recombinant live attenuated T. gondii or a STAg preparation of wild-type or recombinant T. gondii, to a mammal after microbial infection, e.g., up to about 48 hours, about 96 hours, or longer after microbial infection, inhibits the microbial infection, e.g., symptoms thereof, and may also reduce the mortality rate in treated mammals infected with an otherwise lethal dose of microbes. Moreover, surprisingly, inoculation of a mammal with a live attenuated T. gondii up to four months prior to exposure to influenza virus provides protection against influenza infection. In addition, a composition of the invention may protect or lead to an enhanced immune response in a mammal infected with a microbe from later re-infection with the same or a different microbial pathogen.

In one embodiment, the compositions of the invention include extracts (e.g., STAg) from attenuated or avirulent T. gondii, wild-type T. gondii or recombinant T. gondii which expresses heterologous gene products of a microbial pathogen, or live avirulent T. gondii, in an amount effective to modulate an immune response. The T. gondii may be grown in a susceptible tissue culture and isolated therefrom for use in the compositions or methods, or may be used to prepare a soluble antigen extract thereof. The T. gondii used to prepare the soluble antigen extract-containing compositions, such as STAg preparations, can be obtained from any suitable source. In one embodiment, the T. gondii used to prepare a soluble antigen extract-containing composition is a wild-type T. gondii. In another embodiment, the T. gondii used to prepare a soluble antigen extract-containing composition is a live attenuated or avirulent strain of T. gondii. Suitable strains of attenuated or avirulent T. gondii are known in the art and include, for example, strains derived from PruΔ (a hypoxanthine-xanthine-guanosine phosphoribosyl transferase deletion strain), such as N28E2, 73F9, as well as others described in Frankel et al. (2007), the disclosure of which is incorporated by reference herein (see Table 1). Table 1 lists 39 mutants with about 10-fold reduction in the number of cysts per brain compared with infections with wild-type parasites. The second column contains the percentage of cysts per brain for each mutant compared with wild-type (% WT), along with the number of mice infected with each mutant in parenthesis. For the 24F9 mutant, the mice infected with wild-type parasites died during acute infection for that experiment, thus, the number of cysts per brain for wild-type was estimated at 10,000 (typical for wild-type infections). The attenuated strain 73F9 is a Type II strain that was deposited on behalf of Wisconsin Alumni Research Foundation with the American Type Culture Collection (ATCC), 10801 University Blvd, Manassas, Va. 20110-2209, on Jun. 11, 2009 and in the acknowledgement of receipt dated Jun. 11, 2009 was identified as Toxoplasma gondii; 73FI [sic]. A corrected receipt, specifying that one of the deposits was Toxoplasma gondii; 73F9, was received on Jul. 24, 2009. The deposit included human foreskin fibroblasts infected with strain 73F9. The accession number for 73F9 is PTA-10117. The mutant strain of T. gondii N28E2 is a Type II strain that was deposited with the ATCC on Jun. 11, 2009. The deposit included human foreskin fibroblasts infected with strain N28E2. The accession number for N28E2 is PTA-10118.

TABLE 1

| | % WT | | | |
|---|---|---|---|---|
| | (no. of | | | |
| Mutant | mice) | Chromosome | Location | annotation |
| 71C2 | 6 (3) | Ia | 411900 | |
| 9xG4 | 7 (7) | Ia | 599455 | |
| 101F9 | 5 (2) | III | 1202200 | ribosomal protein L15 |
| | | | | putative |
| 57G3 | 9 (2) | IV | 2003950 | |
| 26F7 | 5 (2) | IV | 1407661 | |
| 73F9 | 7 (6) | V | 1367207 | regulator of |
| | | | | chromosome |
| | | | | condensation related |
| 88E8 | 5 (11) | V | 1009640 | |
| 9B5 | 10 (2) | VI | 178451 | |
| 19C3 | 4 (2) | VI | 178451 | |
| 29C3 | 3 (2) | VI | 66771 | family domain- |
| | | | | containing |
| | | | | transmembrane protein |
| 98F7 | 4 (2) | VI | 2577040 | PX domain-containing |
| | | | | protein |
| 12F7 | 7 (6) | VIIa | 1175720 | cyclophilin lysyl- |
| | | | | tRNA synthetase |
| 38C3 | 3 (3) | VIIa | 2463700 | |
| KMME6 | 5 (12) | VIIb | 1785558 | |
| 13E10 | 9 (2) | VIIb | 3944360 | synaptobrevin-like |
| | | | | protein, L-type amino |
| | | | | acid transporter- |
| | | | | related |
| 68C6 | 4 (4) | VIII | 5874600 | |
| 64F2 | 14 (1) | IX | 1352099 | acyl-CoA |
| | | | | dehydrogenase, |
| | | | | short/branched chain |
| | | | | specific, |
| | | | | mitochondrial |
| | | | | (precursor) |
| 90B2 | 33 (2) | IX | 1352051 | acyl-CoA |
| | | | | dehydrogenase, |
| | | | | short/branched |
| | | | | chain specific, |
| | | | | mitochondrial |
| | | | | (precursor) |
| 91G8 | 3 (2) | IX | 3220845 | |
| PRD4 | 3 (12) | IX | 5321038 | patatin-like |
| | | | | phospholipase |
| | | | | domain containing |
| | | | | protein |
| 49E10 | 7 (4) | X | 3362809 | elongation factor G |
| | | | | putative |
| 56C2 | 7 (2) | X | 1882762 | |
| 44C6 | 5 (2) | X | 1666204 | |
| 97F9 | 10 (2) | X | 5856020 | ImpB/MucB/SamB |
| | | | | family domain- |
| | | | | containing protein |
| 41E2 | 0.2 (4) | XI | 2191215 | |
| 40E4 | 6 (4) | XII | 5779451 | |
| 95C5 | 8 (2) | XII | 5331130 | |
| 42F7 | 11 (2) | XII | 109850 | staphylococcal |
| | | | | nuclease |
| | | | | homologue domain- |
| | | | | containing protein |
| 24F9 | 8 (2) | XII | 4733853 | |
| 39B2 | 6 (2) | XII | 78139 | small nuclear |
| | | | | ribonucleoprotein |
| | | | | (snRNP) |
| 40F3 | 3 (2) | XII | 327168 | |
| 40B2 | 6 (2) | XII | 327168 | |
| 18C5 | 6 (3) | TGG994746 | 45323 | SPX domain- |
| | | | | containing protein |
| 7xC11 | 5 (9) | TGG994767 | 15632 | |
| 26G5 | 8 (4) | | | |
| 37B11 | 7 (3) | | | |
| 87D10 | 1 (2) | | | |
| 37B2 | 5 (2) | | | |
| 91E4 | 1 (2) | | | |

The compositions may be produced by growing T. gondii under artificial conditions, for example in tissue cultures, such as described in the examples of the present disclosure, or in vivo in felines. In one exemplary embodiment, T. gondii strains are propagated in human foreskin fibroblast monolayers or in other susceptible tissue cultures, under standard conditions, e.g., at 37° C. with 5% CO₂. A susceptible tissue culture is intended to include a tissue culture that, when inoculated with T. gondii, is able to grow the parasite tachyzoites. Other non-limiting examples of suitable tissue cultures that may be used to grow T. gondii include any nucleated cell such as Vero cells, Chinese hamster ovary (CHO) cells, RAW 264.7 cells (a mouse macrophage cell line), and the like.

A T. gondii culture may be regularly tested for mycoplasma contamination to ensure it is substantially mycoplasma-free. Testing for mycoplasma contamination may be done using any suitable method. Kits for testing cultures for mycoplasma contamination are also commercially available. Examples of such kits include the MycoAlert® Mycoplasma Detection Kit, available from Lonza, Inc. (Basel, Switzerland).

T. gondii tachyzoites may be formulated into the compositions of the invention. The culture of T. gondii may be provided in a purified or an unpurified form. In some embodiments, the T. gondii tachyzoites may be purified or partially purified from the host cells prior to formulating the T. gondii into the composition. This may be achieved by lysing the host cells using any suitable technique, such as syringe lysing.

As noted above, in one particular embodiment, the composition comprises STAg. STAg-containing preparations may be prepared by any conventionally known technique. In one particular example, STAg is prepared as described in the examples set forth herein. Briefly, T. gondii is grown under standard conditions, as described above. When the parasites begin to lyse the host cells, monolayers are scraped, syringed passed through a needle (e.g., 27-gauge), pelleted via centrifugation (e.g., at 100,000×g), washed (e.g., with PBS), and resuspended. The STAg preparation may then be prepared by sonicating the T. gondii suspension under suitable conditions (e.g., with five 30 second pulses). Other procedures for preparing STAg preparations are known in the art. Advantageously, the STAg-containing preparations are non-infectious. As such, STAg may be prepared from wild-type T. gondii, as well as from recombinant, e.g., avirulent, live T. gondii. Any art-known technique may be used to produce a mutated or genetically modified T. gondii strain that is avirulent, including but not limited to chemical mutagenesis and genetic engineering. Examples of suitable avirulent strains include the above-described 73F9 and N28E2 T. gondii strains.

For compositions comprising inactivated T. gondii, the T. gondii may be inactivated using any art-known technique including, for example, contact with an inactivating agent such as formalin, beta-propiolactone (BPL), heat, binary ethylenimine (BEI), detergents, or subjecting the culture to freeze/thaw. Any suitable process may be used for heat-killing the T. gondii. In one non-limiting example, the T. gondii may be heat killed by subjecting the T. gondii to temperatures of from about 95° C. to about 100° C. for about 5 minutes.

In one embodiment, a composition of the invention comprises one or more isolated proteins, such as isolated lipoxygenase, recombinant T. gondii protein(s), or recombinant cells expressing one or more Toxoplasma proteins, including wild-type or mutant proteins, e.g., those having altered activity and/or stability, as well as splicing variants, in an amount effective to elicit an antiinfective or antimicrobial response. In one embodiment, the composition modulates the host immune system, thereby allowing for increased ability to ward off infective microbes or protection from pathological immune activation. For instance, recombinant protein may be isolated from a suitable expression system, such as bacteria, insect cells or yeast, e.g., E. coli, L. lactis, Pichia or S. cerevisiae or other bacterial, insect or yeast expression systems, or mammalian expression systems such as T-REx™ (Invitrogen), and isolated protein may be obtained from the native organism, e.g., lipoxygenase may be isolated from a plant or T. gondii. For example, to prepare isolated recombinant T. gondii proteins, any suitable host cell may be employed, e.g., E. coli or yeast, to express those proteins. Those cellular expression systems may also be employed as delivery systems, e.g., E. coli or L. lactis, expressing a heterologous lipoxygenase, such as one expressed on the cell surface or in a secreted form. A suitable cellular delivery system may be one for oral delivery. The recombinant protein useful in the compositions and methods of the invention may be expressed on the surface of a prokaryotic or eukaryotic cell, or may be secreted by that cell, and may be expressed as a fusion, e.g., for targeting, for instance, the recombinant protein may be fused to an antibody an antibody or a portion of an antibody, e.g., ScFv or Fc such as a mutant Fc that stabilizes the fusion, or a cell-surface molecule specific for a type of cell, for instance a neutrophil, for purification, e.g., a His tag may be fused to the recombinant protein, or the recombinant protein may be fused to a molecule with a distinct function, e.g., an immune response stimulator, such as an adjuvant, an immune response inhibitor, or the recombinant protein may be modified to alter MHC binding determinants, T-cell receptors, B-cell receptors or antigenic epitopes, or may be linked to a molecule that alters solubility (e.g., prevents aggregation) or half-life, e.g., a PEGylated molecule, of the resulting chimeric molecule. In one embodiment, the composition of the invention may comprise a recombinant cell expressing one or more recombinant T. gondii proteins, e.g., on the cell surface or as a secreted protein, or a recombinant T. gondii modified to express one or more heterologous gene products, e.g., proteins expressed by a different pathogen (see, e.g., Charest et al., 2000, which discloses vectors useful to express heterologous gene products in T. gondii).

In one embodiment, the present invention is directed to a method of treating, inhibiting or preventing a viral infection in a mammal, e.g., viruses including but not limited to rabies, influenza A, influenza B, influenza C, flaviviruses including West Nile virus and Dengue virus, paramyxoviruses including Respiratory Syncyctial virus, parvoviruses, retroviruses, and gastroenteroviruses including rotavirus, norovirus, and astrovirus. In one embodiment, the method comprises administering an amount of a composition of the invention, e.g., after the mammal has been infected with or exposed to a virus, effective to inhibit or treat the viral infection. In one embodiment, the composition comprises a soluble antigen extract from T. gondii tachyzoites, e.g., from a live avirulent T. gondii. In another embodiment, the invention generally relates to vaccines and methods for immunizing a mammal against viral infection. In one embodiment, a composition of the invention is administered to a mammal before the mammal is exposed to the virus. In one embodiment, the pathogen is an influenza virus. In one embodiment, the influenza virus is a H5N1 virus.

In one embodiment, the invention provides a method of treating, inhibiting or preventing a parasite infection in a mammal, e.g., infection by various species of Plasmodium, such as Plasmodium berghei, and Plasmodium falciparum and other Coccidia such as Cryptosporidium parvum, or other protozoan parasites such as Trypanosome brucei, Entamoeba histolytica, Leishmania species and helminth parasites such as Schistosoma mansoni. In one embodiment, the method comprises administering an amount of a composition of the invention to the mammal effective to inhibit or treat the parasitic infection after the mammal has been infected with the parasite. In one embodiment, the composition comprises a soluble antigen extract from T. gondii tachyzoites. In one embodiment, a composition of the invention is administered to a mammal before the mammal is exposed to the parasite.

In one embodiment, the invention provides a method of treating, inhibiting or preventing a bacterial infection, e.g., infection by Listeria or a pan resistant gram-negative bacilli, such as Pseudomonas aeruginosa, or multi-resistant gram-positive bacteria like methicillin resistant Staphylococcus aureus, as well as Mycobacterium tuberculosis, or nontuberculosis Mycobacterium or Nocardia, in a mammal. In one embodiment, the method comprises administering an effective amount of a composition of the invention to the mammal after the mammal has been infected with the bacterium. In one embodiment, the composition comprises a soluble antigen extract from T. gondii tachyzoites. In another embodiment, the composition comprises isolated lipoxygenase, either native or recombinant, from one or more sources, including plant, bacterial or parasite sources. In one embodiment, the method comprises administering an effective amount of a composition of the invention to the mammal before the mammal is infected with the bacterium.

In one embodiment, the invention provides a method of treating, inhibiting or preventing a fungal infection in a mammal, e.g., Cryptococcus, Aspergillus, species, Histoplasma capsulatum, Blastomyces dermatitidis, Coccidiomycosis immitis and Penicillium marcenscens. In one embodiment, the method comprises administering an effective amount of a composition of the invention to the mammal after the mammal has been infected with the fungus. In one embodiment, the composition comprises a soluble antigen extract from T. gondii tachyzoites. In one embodiment, the method comprises administering an effective amount of a composition of the invention to the mammal before the mammal is infected with the fungus.

As will be apparent to one skilled in the art, the optimal concentration of the T. gondii or soluble extract thereof, recombinant cell or isolated protein in the composition will necessarily depend upon the specific immunomodulatory agent(s) used, e.g., heat-killed or live avirulent T. gondii or extracts thereof, the characteristics of the mammal, the type and amount of adjuvant, if any, and the nature of the microbial infection. These factors can be determined by those of skill in the medical and pharmaceutical arts in view of the present disclosure. In general, the active agent(s) in the composition of the invention are administered at a concentration that either modulates antimicrobial activity against microbial infection or modulates an immune response allowing the host to recover from or clear a microbial infection, without significant, harmful or adverse side effects.

Specific dosages may be adjusted depending on conditions of disease, the age, body weight, ethnic background, general health conditions, sex, diet, lifestyle and/or current therapeutic regimen of the mammal, as well as for intended dose intervals, administration routes, excretion rate, and combinations of drugs. Any of the dosage forms described herein containing effective amounts are well within the bounds of routine experimentation and therefore, well within the scope of the instant disclosure.

A composition comprising a soluble extract preparation may comprise protein in an amount of from about 100 μg per mL to about 1000 μg per mL, in some instances from about 200 μg per mL to about 1000 μg per mL, and in some instances from about 500 μg per mL to about 1000 μg per mL. In one embodiment, the composition comprises a soluble extract in an amount of about 200 μg of STAg protein per dose for a mammal weighing about 20 to 25 g. In one embodiment, for larger mammals, e.g., humans, the composition comprises a soluble extract in an amount of about 10 mg to about 1000 mg, e.g., about 20 mg to about 500 mg, or for smaller mammals, e.g., mice, the composition comprises a soluble extract in an amount of about 100 to 1000 μg, e.g., 200 μg.

The desired dose of the composition may be presented in a continuous infusion, a single dose, or as divided doses administered at appropriate intervals, for example as two, three, four or more sub-doses per day. Optionally, a dose of composition may be administered on one day, followed by one or more booster doses spaced as desired thereinafter. In one exemplary embodiment, an initial dose is given, followed by a boost of the same composition approximately two to four days later. In one particular embodiment, the mammal is administered a first dose of the composition at about 48 hours post-infection and a second dose of the composition at about 96 hours post-infection. Other dosage schedules may also be used, e.g., prophylactic use during an outbreak or pandemic to decrease morbidity post infection.

Following an initial administration of the composition, mammals may receive one or several booster doses adequately spaced thereafter. In some embodiments, the booster doses comprise the same amounts and type of active agent as the initial administration. In other embodiments, the booster doses may comprise a reduced amount and/or a different type of active agent, for instance, the original inoculum may include STAg of a live avirulent T. gondii and the booster may be isolated (native or recombinant) lipoxygenase.

In addition to the T. gondii culture, STAg, recombinant cells or isolated protein, the composition of the invention may further comprise one or more suitable pharmaceutically acceptable carriers. As used herein, the term “pharmaceutically acceptable carrier” refers to an acceptable vehicle for administering a composition to mammals comprising one or more non-toxic excipients which do not react with or reduce the effectiveness of the pharmacologically active agents contained therein. The proportion and type of pharmaceutically acceptable carrier in the composition may vary, depending on the chosen route of administration. Suitable pharmaceutically acceptable carriers for the compositions of the present disclosure are described in the standard pharmaceutical texts. See, e.g., “Remington's Pharmaceutical Sciences”, 18^{th} Ed., Mack Publishing Company, Easton, Pa. (1990). Specific non-limiting examples of suitable pharmaceutically acceptable carriers include water, saline, dextrose, glycerol, ethanol, or the like and combinations thereof.

Optionally, the composition may further comprise minor amounts of auxiliary substances such as agents that enhance the antimicrobial effectiveness of the preparation, stabilizers, preservatives, and the like.

In one embodiment, the composition may also comprise a bile acid or a derivative thereof, in particular in the form of a salt. These include derivatives of cholic acid and salts thereof, in particular sodium salts of cholic acid or cholic acid derivatives. Examples of bile acids and derivatives thereof include cholic acid, deoxycholic acid, chenodeoxycholic acid, lithocholic acid, ursodeoxycholic acid, hyodeoxycholic acid and derivatives such as glyco-, tauro-, amidopropyl-1-propanesulfonic-, amidopropyl-2-hydroxy-1-propanesulfonic derivatives of the aforementioned bile acids, or N,N-bis (3Dgluconoamidopropyl) deoxycholamide. A particular example is sodium deoxycholate (NaDOC).

Examples of suitable stabilizers include protease inhibitors, sugars such as sucrose and glycerol, encapsulating polymers, chelating agents such as ethylene-diaminetetracetic acid (EDTA), proteins and polypeptides such as gelatin and polyglycine and combinations thereof.

Optionally, the composition may further comprise an adjuvant in addition to the T. gondii, STAg, recombinant cells or isolated protein described herein. Suitable adjuvants for inclusion in the compositions of the present disclosure include those that are well known in the art, such as complete Freund's adjuvant (CFA) that is not used in humans, incomplete Freund's adjuvant (IFA), squalene, squalane, alum, and various oils, all of which are well known in the art, and are available commercially from several sources, such as Novartis (e.g., Novartis' MF59 adjuvant).

Depending on the route of administration, the compositions may take the form of a solution, suspension, emulsion, or the like. A composition of the invention can be administered intranasally or through enteral administration, such as orally, or through subcutaneous injection, intra-muscular injection, intravenous injection, intraperitoneal injection, or intra-dermal injection to a mammal, e.g., humans, horses, other mammals, etc. Compositions may be formulated for a particular route of delivery, e.g., formulated for oral delivery.

For parenteral administration, the composition of the invention may be administered by intravenous, subcutaneous, intramuscular, intraperitoneal, or intradermal injection, and may further comprise pharmaceutically accepted carriers. For administration by injection, the composition may be in a solution in a sterile aqueous vehicle which may also contain other solutes such as buffers or preservatives as well as sufficient quantities of pharmaceutically acceptable salts or of glucose to make the solution isotonic.

The composition may be delivered to the respiratory system, for example to the nose, sinus cavities, sinus membranes or lungs, in any suitable manner, such as by inhalation via the mouth or intranasally. The composition may be dispensed as a powdered or liquid nasal spray, suspension, nose drops, a gel or ointment, through a tube or catheter, by syringe, by packtail, by pledget, or by submucosal infusion. The composition may be conveniently delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the composition and a suitable powder base such as lactose or starch. Examples of intranasal formulations and methods of administration can be found in PCT publications WO 01/41782, WO 00/33813, and U.S. Pat. Nos. 6,180,603; 6,313,093; and 5,624,898, all of which are incorporated herein by reference and for all purposes. A propellant for an aerosol formulation may include compressed air, nitrogen, carbon dioxide, or a hydrocarbon based low boiling solvent. The composition of the invention may be conveniently delivered in the form of an aerosol spray presentation from a nebulizer or the like. In some aspects, the active ingredients (i.e., T. gondii cultures, STAg proteins) are suitably micronized so as to permit inhalation of substantially all of the active ingredients into the lungs upon administration of the dry powder formulation, thus the active ingredients will have a particle size of less than 100 microns, desirably less than 20 microns, and preferably in the range 1 to 10 microns. In one embodiment, the composition is packaged into a device that can deliver a predetermined, and generally effective, amount of the composition via inhalation, for example a nasal spray or inhaler.

### Vaccines

Without wishing to be bound to any particular theory, it is believed that inoculation or immunization of a mammal with T. gondii prior to exposure to a microbe may boost the immune response, which in turn enhances protection against subsequent microbial infection. In one embodiment, the innate immune response may increase the amount of neutrophils present in the mammal, which may lower microbe titers, and/or may increase adaptive immune responses such as heterologous antigen-specific CD8⁺ T cell proliferation, as compared to mammals not pre-inoculated with T. gondii. These T cells proliferate upon exposure to an immunogen, thus providing host immune memory against the microbe. Other mechanisms of protection may also be involved.

Thus, a composition of the invention may further contain a microbial immunogen that is capable of eliciting an adaptive immune response against the microbe. In one embodiment, the microbial immunogen may be conjugated chemically or recombinantly to a T. gondii protein or a lipoxygenase from any source. For example, a microbial immunogen may be expressed as a fusion with a lipoxygenase and the resulting fusion protein may alter both innate and adaptive immune responses. Therefore, the compositions of the invention may be employed as a vaccine when employed with one or more immunogens of a microbe. Advantageously, those vaccines may prevent infection and/or limit the severity of the infection, or otherwise enhance the adaptive immune response relative to a vaccine that does not include T. gondii, STAg, recombinant cells or isolated protein, e.g., isolated lipoxygenase. In one embodiment, the immunogen is a protein, e.g., a recombinant protein, peptide or polysaccharide, glycoprotein or lipopolysaccharide. In another embodiment, the immunogen may be DNA molecules (polynucleotides) which produce the antigen in cells after introduction of the DNA molecule to the cells (e.g. by transfection). The immunogen may be, for example, a live attenuated microbial pathogen, one or more proteins of the microbe, or a combination thereof. For example, the immunogen may be split virus antigens, subunit antigens (either recombinantly expressed or prepared from whole virus), and/or inactivated whole virus which may be chemically inactivated by any suitable means including, for example, by treating with formaldehyde, formalin, β-propiolactone, or otherwise inactivated such as by ultraviolet or heat inactivation. The immunogen may be provided in a purified or an unpurified form.

The vaccines of the present disclosure may further comprise one or more suitable pharmaceutically acceptable carriers. As used herein, the term “pharmaceutically acceptable carrier” refers to an acceptable vehicle for administering a vaccine to mammals comprising one or more non-toxic excipients which do not react with or reduce the effectiveness of the pharmacologically active agents contained therein. The proportion and type of pharmaceutically acceptable carrier in the vaccine may vary, depending on the chosen route of administration. Suitable pharmaceutically acceptable carriers for the vaccines of the present disclosure are described in the standard pharmaceutical texts. See, e.g., “Remington's Pharmaceutical Sciences”, 18^{th} Ed., Mack Publishing Company, Easton, Pa. (1990). Specific non-limiting examples of suitable pharmaceutically acceptable carriers include saline (e.g., PBS), dextrose, glycerol, or the like and combinations thereof.

In addition, if desired, the vaccine can further contain minor amounts of auxiliary substances such as agents that enhance the antiviral effectiveness of the composition, stabilizers, preservatives, and the like.

Depending on the route of administration, the vaccine may take the form of a solution, suspension, emulsion, or the like. A vaccine of the present disclosure can be administered orally, intranasally, or through parenteral administration, such as through sub-cutaneous injection, intra-muscular injection, intravenous injection, intraperitoneal injection, or intra-dermal injection to a mammal, e.g., humans, horses, other mammals, etc. Typically, the vaccine is administered through intramuscular or intradermal injection.

For parenteral administration, the vaccines of the present disclosure may be administered by intravenous, subcutaneous, intramuscular, intraperitoneal, or intradermal injection, which optionally may further comprise pharmaceutically accepted carriers. For administration by injection, the vaccine may be a solution in a sterile aqueous vehicle which may also contain other solutes such as buffers or preservatives as well as sufficient quantities of pharmaceutically acceptable salts or of glucose to make the solution isotonic.

The vaccine may be delivered locally to the respiratory system, for example to the nose, sinus cavities, sinus membranes or lungs, in any suitable manner, such as by inhalation via the mouth or intranasally. The vaccines can be dispensed as a powdered or liquid nasal spray, suspension, nose drops, a gel or ointment, through a tube or catheter, by syringe, by packtail, by pledget, or by submucosal infusion. The vaccines may be conveniently delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the vaccine and a suitable powder base such as lactose or starch. Examples of intranasal formulations and methods of administration can be found in PCT publications WO 01/41782, WO 00/33813, and U.S. Pat. Nos. 6,180,603; 6,313,093; and 5,624,898, all of which are incorporated herein by reference and for all purposes. A propellant for an aerosol formulation may include compressed air, nitrogen, carbon dioxide, or a hydrocarbon based low boiling solvent. The vaccines of the present disclosure can be conveniently delivered in the form of an aerosol spray presentation from a nebulizer or the like. In some aspects, the active ingredients (i.e., T. gondii cultures) are suitably micronized so as to permit inhalation of substantially all of the active ingredients into the lungs upon administration of the dry powder formulation, thus the active ingredients will have a particle size of less than 100 microns, desirably less than 20 microns, and preferably in the range 1 to 10 microns. In one embodiment, the vaccine is packaged into a device that can deliver a predetermined, and generally effective, amount of the vaccine via inhalation, for example a nasal spray or inhaler.

The vaccines of the present disclosure are administered prophylactically. For instance, administration of the vaccine may be commenced before or at the time of infection. In particular, the vaccines may be administered up to about 1 month or more, or more particularly up to about 4 months or more before the mammal is exposed to the microbe. Optionally, the vaccines may be administered as soon as 1 week before infection, or more particularly 1 to 5 days before infection.

The desired vaccine dose may be presented in a single dose or as divided doses administered at appropriate intervals, for example as two, three, four or more sub-doses per day. Optionally, a dose of vaccine may be administered on one day, followed by one or more booster doses spaced as desired thereinafter. In one exemplary embodiment, an initial vaccination is given, followed by a boost of the same vaccine approximately one week to 15 days later.

### Pharmaceutical Formulations

The compositions of this invention may be formulated with conventional carriers and excipients, which will be selected in accord with ordinary practice. Aqueous formulations are prepared in sterile form, and when intended for delivery by other than oral administration, will generally be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone they may be present as pharmaceutical formulations. The formulations, both for veterinary and for human use, of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

Pharmaceutical formulations according to the present invention may include one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

The amount of active ingredient that may be combined with the carrier material to produce a single dosage form will vary depending upon the host treated and the particular mode of administration.

For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for intrapulmonary or nasal administration may have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of a given condition.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Exemplary unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can also be formulated to provide controlled release of the active ingredient to allow less frequent dosing or to improve the pharmacokinetic or toxicity profile of the active ingredient. Accordingly, the invention also provided compositions comprising one or more compounds of the invention formulated for sustained or controlled release.

An effective dose of an active ingredient depends at least on the nature of the condition being treated, toxicity, whether the active ingredient is being used prophylactically (e.g., lower doses may be employed), the method of delivery, and the pharmaceutical formulation, and will be determined by the clinician using conventional dose escalation studies. It can be expected to be from about 0.0001 to about 100 mg/kg body weight per day. Typically, from about 0.01 to about 10 mg/kg body weight per day. More typically, from about 0.01 to about 5 mg/kg body weight per day. More typically, from about 0.05 to about 0.5 mg/kg body weight per day. For example, the daily candidate dose for an adult human of approximately 70 kg body weight will range from 1 mg to 1000 mg, e.g., from 5 mg to 500 mg, and may take the form of single or multiple doses. For instance, about 10 mg to about 750 mg, e.g., about 20 mg to about 500 mg, or any integer in between, of the active ingredient may be administered to a human (see Reagsn-Shaw et al., 2008).

The invention will be described by the following nonlimiting examples.

### Example I

### Use of T. gondii Preparations to Inhibit or Treat Influenza

### Methods

Laboratory facilities. All experiments using H5N1 viruses, including work with animals, were performed in an enhanced biosafety level 3 (BSL3+) containment laboratory approved by the Centers for Disease Control and Prevention and the U.S. Department of Agriculture Select Agent program. Investigators were required to wear appropriate personal respirator equipment (RACAL; Health and Safety Inc., Frederick, Md.). Mice were housed in HEPA-filtered negative pressure cages (M.I.C.E. racks; Animal Care Systems, Littleton, Colo.).

T. gondii culture. Propagation of the T. gondii tachyzoites was done in human foreskin fibroblast (HFF) monolayers at 37° C. with 5% CO₂ under standard conditions (Ware & Kasper, 1987) and tested to be mycoplasma-free (Lonza, Switzerland). All attenuated parasites were derived from PruΔ (hypoxanthine-xanthine-guanosine phosphoribosyl transferase deletion) strain. The T. gondii mutant 73F9 was described in Frankel et al. (2007). The N28E2 mutant is under characterization. Heat inactivated T. gondii was prepared by subjecting wild-type T. gondii to temperatures of from about 95° C. to about 100° C. for about 5 minutes.

Viruses, cells, and viral infections. The highly pathogenic avian H5N1 influenza virus A/Hong Kong/483/97 (provided Dr. Alexander Klimov, Centers for Disease Control and Prevention, Atlanta, Ga.) was propagated in Madin Darby canine kidney (MDCK) cells as described in Jones et al. (2006). Viral titers were determined by fifty percent tissue culture infectious dose (TCID₅₀) analysis in MDCK cells (as described in Jones et al., 2006). MDCK cells were cultured in modified Eagle's medium (MEM, CellGro, Herndon, Va.) supplemented with 4.5 g of glucose per liter, 2 mM glutamine, and 10% fetal bovine serum (FBS, Harlan, Madison, Wis.) at 37° C., 5% CO₂.

Chronic infection of T. gondii pre-influenza infection. 7-8 week old C57BL/6J mice (Jackson Laboratory, Bar Harbor, Me.) were intraperitoneally (i.p) inoculated with differing numbers of wild-type (WT), heat-inactivated WT (HI), or the attenuated mutant strains of T. gondii 73F9 (Frankel et al., 2007) or N28E2, grown under standard conditions (HFFs in DMEM with 10% fetal bovine serum, pH 7.2, 37° C. in 5% CO₂). After 1 or 4 months, mice were transported into a CDC-APHIS approved BSL-3 enhanced laboratory, lightly anesthetized, and intranasally infected with 10⁴ TCID₅₀ (1 mouse lethal dose₅₀ [MLD₅₀]) A/Hong Kong/483/97 (HK/483) H5N1 influenza virus. Mice were monitored daily for weight loss and clinical signs of infection (Morton et al., 2000). At 0, 3 and 7 days post influenza infection (dpi), sera and tissues were collected from 2 controls and 3 infected mice from each group and monitored for viral titers (Jones et al., 2006), cytokine levels by ELISA (Tumpey et al., 2000) and flow cytometry (Tumpey et al., 2000).

STAg preparation. Unless otherwise indicated, STAg was prepared from N28E2 parasites grown in human foreskin fibroblast (HFF) cells under standard cell culture conditions and shown to be mycoplasma-free (Lonza, Switzerland). Briefly, when the parasites were beginning to lyse the host cells, monolayers were scraped, passed twice through a 27-gauge needle, and pelleted at 420×g. Parasites were then washed in PBS without divalent cations, and resuspended to 4×10⁸ parasites per mL. After sonication with five 30 second pulses, parasites were centrifuged at 100,000×g for 45 minutes and supernatants collected and stored at −70° C. until use. An equal number of flasks containing HFF cells without parasites were processed under exact conditions and resuspended to the same volume to be used as the HFF control.

STAg treatments of influenza infected mice. Four to 6 week old C57BL/6J or B6.129S7-Rag1^{tm1Mom}/J (Jackson Laboratory, Bar Harbor, Me.) mice were infected with influenza virus as described above and at 48 hours post infection (hpi) (1 treatment) and 96 hpi (2 treatments) mice were intravenously (i.v.) administered 200 μL per mouse PBS, HFF preparation (negative control), or STAg and monitored as described above.

Flow Cytometry. Flow cytometry on lung homogenates was performed as described in Tumpey et al. (2000). Briefly, lungs were dissected, lightly minced, and washed in cold PBS. Pooled lungs from each experimental group were placed into RPMI-1640 (Mediatech, Herndon, Va.) supplemented with 2 mg/mL collagenase B (Roche) and single cell populations generated by pushing homogenates through 70 μm cell strainers (BD Falcon). After centrifugation, the cell pellet was resuspended in red blood cell (RBC) lysis solution, washed, cell number quantitated, and 1.0×10⁶ cells per group stained blocked with 10% normal rat serum (eBioscience) for 30 minutes at 4° C. Cells were then stained with LIVE/DEAD® fixable dead cell staining (Invitrogen) followed by specific staining for different cell populations. 10,000 total live cell events were gated per sample on a BD LSRII flow cytometer. Percent positive were compared to total gated events by FlowJo® Flow Cytometry Analysis Software.

Quantification of Brain Cysts. Brain cysts were quantified as described in Mordue et al. (2007).

Histopathologic analysis. Tissues were collected and fixed in 10% neutral buffered formalin solution, processed, and paraffin embedded. Histopathologic examination was performed by using hematoxylin- and eosin (H&E)-stained sections.

Statistical analysis. Statistical significance of the data was determined by using analysis of variance (ANOVA) or Student's t-test on Graph Pad Prism (San Diego, Calif.). Results are representative of at least 3 separate experiments with at least 3 mice per group.

### Results

T. gondii is an obligate intracellular parasite of any warm-blooded animal that has worldwide distribution. Within the intermediate host, acute infection peaks 7-10 days post infection, after which T. gondii differentiates into a chronic life-long cyst form within the striated muscle and the central nervous system. As this chronic infection with T. gondii was shown to be protective against a lethal challenge of the picornavirus mengo, it was determined if chronic infection with T. gondii would protect mice against a severe influenza infection. Mice were infected with wild type (WT) or attenuated mutants of T. gondii (73F9 and N28E2; Frankel et al., 2007) for one month, then challenged with a lethal dose of highly pathogenic avian H5N1 influenza virus. Mice were monitored daily for weight loss and clinical signs of infection and at 0, 3 and 7 days post influenza (dpi), sera and tissues were collected to determine viral titers and cytokine levels by ELISA (Tumpey et al., 2000).

Eighty percent of the mice infected with WT T. gondii for 1 month survived subsequent influenza challenge as compared to no survivors in the mice not receiving prior T. gondii infection (FIG. 1A). There was no difference in survival or weight loss with differing T. gondii doses; from 50-5000 parasites were protective. However, heat inactivating T. gondii prior to inoculation decreased survival (FIG. 1A) suggesting that viable parasites were required for protection. The T. gondii mutant 73F9 is defective during acute infection and has a reduction in the overall number of cysts in the brain during chronic infection (Frankel et al., 2007). 73F9 was as protective as WT T. gondii, but only when a high inoculum of parasites was used (compare 500 versus 5000 73F9 parasites in FIG. 1A). The mutant N28E2 is similar to WT T. gondii during acute infection, but is defective during chronic infection and unable to persist (unpublished data). While infection with a high inoculum of N28E2 was protective similar to WT T. gondii, a low inoculum of N28E2 was 100% protective with an absence of clinical symptoms (FIG. 1A). At 4 months post-infection with T. gondii (late chronic), mice infected with WT parasites were still protected against influenza infection, while mice infected with 73F9 or N28E2 parasites were not (FIG. 1B). These results again suggest that viable parasites were required for protection and that the 73F9 or N28E2 mutants do not form a persistent life-long infection.

As there are two ways to survive infection, resistance that limits pathogen burden and the quelling of an immune response so as not to overreact to pathogen infection, levels of influenza virus in infected mice were evaluated. By 3 days post-influenza virus infection, titers were already reduced by 4-logs in mice inoculated with the highest concentrations of WT or 73F9 T. gondii (FIG. 1C). At 7 dpi, viral titers in mice inoculated with the highest concentrations of WT or N28E2 were also significantly reduced. By 11 dpi, all surviving mice had cleared the virus from their lungs (data not shown). Similar results were observed with the mice infected for 4 months with WT T. gondii (data not shown). These studies suggest that increased survival can be associated with decreased viral titers. However, low inoculum with the N28E2 mutant consistently afforded the best protection against severe influenza virus infection yet viral titers in the lungs were not significantly lowered (FIGS. 1A and 1C). This result suggests that T. gondii may protect by several different mechanisms dependent and independent of controlling viral replication.

T. gondii is a potent inducer of innate immune cells including macrophages, neutrophils, and NK cells in vitro and in vivo. To define the mechanism of protection, the lungs were isolated on days 0, 3, and 7 post-influenza virus infection in mice pre-infected with T. gondii and monitored for cytokine levels by ELISA. At three dpi, flow cytometric analysis showed that increases in neutrophils correlate with decreases in viral titers (FIGS. 1C and 6B). Previous studies have demonstrated the importance of neutrophils in controlling early viral replication during severe influenza virus infections (Tumpey et al., 2005; Perrone et al., 2005). At seven dpi, increases in CD8⁺ T cells correlate with decreases in viral titers (FIGS. 1C and 6B). Levels of interleukin 1 (IL-1), IL-6, IL-10, tumor necrosis factor-alpha (TNF-α) transforming growth factor-beta (TGF-β), interferon-alpha and interferon-gamma (IFN-γ) were measured; only the levels of IFN-gamma increased significantly at 7 dpi (FIG. 3C). While IFN-gamma is essential for the control of T. gondii infection (Suzuki et al. 1988), susceptibility to influenza A infection is not changed in mice with null mutations in the receptor for IFN-gamma (Price et al., 2000). TGF-beta is induced during T. gondii infection (Bermudez et al., 1993; Hunter et al., 1993), and the present studies show that influenza virus may suppress TGF-beta production (FIG. 3D).

Soluble tachyzoite antigens (STAg) prepared from T. gondii stimulated innate immune cells similar to viable parasites, suggesting that parasite replication was not required. Given the decreased viral titers within 3 dpi, it was hypothesized that T. gondii-mediated protection involved up-regulation of innate immune responses. Mice were infected with a lethal dose of influenza virus, then at 2 dpi (1 treatment) and 4 dpi (2 treatments), mice were intravenously administered PBS, HFF preparation (negative control), or STAg and monitored for weight loss and clinical signs of infection. All of mice treated with PBS or HFF succumbed to influenza infection or had to be euthanized by 12 dpi (FIG. 2A). In contrast, 70% of mice treated with STAg two days after lethal H5N1 influenza virus infection survived (FIG. 2A). Similar results were observed with a single dose of STAg. Increased survival was accompanied by decreased viral titers (FIG. 2B). Lung titers in the virus alone group were about 10^{5.5} TCID₅₀/ml on days 5 and 8 post-infection. At 5 dpi (2 days post 1-treatment), HFF had no effect on lung titers; however at 8 dpi (2-treatments) HFF-treated mice had lower titers as compared to untreated mice (p<0.01, FIG. 2B).

In contrast, STAg treatment dramatically decreased viral titers at both times with lung titers below the limit of detection at 5 dpi (p<0.001, FIG. 2B), and at 8 dpi, titers were still significantly decreased as compared to untreated influenza virus infected mice (p<0.003). When the STAg-treated survivors were re-challenged with 10-fold more HK/483 influenza virus (10 MLD₅₀) 21 days post completion of the initial experiment, there was complete protection (FIG. 2G). Actually, the STAg-treated survivors did not even lose weight upon re-challenge (FIG. 2H). Thus, in spite of the dramatic decrease in viral titers, STAg triggers an innate immune or inflammatory responses that lead to enhanced adaptive responses. In the virus alone mice, there was reduction in IFN-gamma by 7 dpi likely correlating with the lack of adaptive immune response, which is a hallmark of the severe H5N1 influenza virus infections (Maines et al., 2008). Lymphopenia is pronounced in humans and animals with fatal H5N1 influenza virus infection (La Gruta et al., 2007).

These studies show that a single administration of STAg two days after a severe influenza virus infection was protective. Further, there was a significant decrease in viral titers. The STAg-treated mice were protected against re-infection with a 10× lethal dose of avian influenza 21 days after the initial infection. These results may indicate that the STAg-containing preparations help mice survive to develop a protective adaptive immune response, e.g., the STAg-containing preparations work as an adjuvant by driving an innate immune response that is protective, so that an adaptive immune response can develop and provide long-term protection. Alternatively, the STAg-containing preparations may enhance adaptive immunity.

Histopathologic analysis on the lungs of STAg, HFF, or virus alone control mice were performed on day 5 post infection. As can be seen from FIGS. 2C-F, mice administered a double dose of the STAg preparation had less inflammation in the bronchium and alveolum as compared to mice administered a double dose of the PBS or HFF controls. These studies show that a STAg administration two days after a lethal influenza virus infection was protective and significantly decreased viral titers and lung inflammation.

In spite of the dramatic decrease in viral titers, there is sufficient induction of a specific immune response to drive memory and protection from subsequent infection. These are the first studies to describe a parasite extract that generates a protective and potentially lasting immune response against severe influenza virus infections. Current antiviral therapies must be administered within 36 to 48 hpi to be effective and require continual dosing. Given that STAg involves a general, immune-driven protective host response, these compositions advantageously have potential for efficacy when administered more than 48 hours after viral infection. Additionally, as efficacy entails a general immune response, it is less likely to drive viral resistance than agents targeted specifically at viral proteins.

To examine the mechanism of STAg protection, the lungs were isolated on days 5 and 8 post-influenza infection in STAg-treated mice and monitored for cytokine levels by ELISA. Only the levels of IFN-α (FIG. 3A), IFN-γ (FIG. 3B) and TGF-β (FIG. 3D) changed significantly. IFN-γ levels were increased in the lungs of infected mice on days 5 and 8 post-influenza infection as compared to uninfected mice (FIG. 3A). Administration of either HFF or STAg led to elevated IFN-γ levels at 5 days post-influenza infection; however only the STAg treated mice had increased survival and reduced viral titers (FIGS. 2A and B) suggesting that protection may be independent of type 1 IFNs. Future studies in type 1 IFN knockout mice will directly address this question.

In contrast to the type 1 IFNs, there was a dramatic increase in IFN-γ levels in the lungs of STAg treated mice on days 5 and 8 post-viral infection as compared to uninfected controls, virus alone, or influenza infected mice treated with HFF (FIG. 3B). By day 5 post-influenza infection (mice received 2 doses of STAg), lung IFN-γ levels increased from 124 pg/mL to 2789 pg/mL (22-fold increase) elevating to a 28-fold increase by 8 days post-viral infection. Although not as dramatic, pre-infecting mice with T. gondii prior to viral infection also led to elevated levels of IFN-γ in the lungs (FIG. 3C). Even 1 month pre-viral infection, WT T. gondii infection led to elevated IFN-γ levels within 3 days after viral infection (FIG. 3C); while the mutant attenuated T. gondii strains were a bit delayed. Lung IFN-γ levels did not increase above virus alone levels until 7 dpi. IFN-γ is produced primarily by natural killer (NK) and natural killer T (NKT) cells as part of the innate immune response, and by Th1 CD4⁺ and CD8⁺ cytotoxic T lymphocyte effector T cells once antigen-specific immunity develops (Schoenborn and Wilson, 2007). Given the timing of the response, it was hypothesized that T cells were not involved in the increased IFN-γ or protection afforded by STAg treatment.

To test this, RAG^{-/-} mice were infected with influenza virus and treated with STAg 2 days post-infection as described and monitored for morbidity. Fifty percent of the RAG^{-/-} treated with STAg survived infection (FIG. 4A) compared to 80% of wild-type mice (FIG. 2A). However, unlike WT mice where STAg treatment reduced viral titers within 5 dpi (FIG. 2B), there was no reduction in viral titers in STAg-treated RAG^{-/-} mice as compared to untreated infected mice (FIG. 4B). In fact, by 8 dpi, viral titers were significantly increased in the STAg treated mice (p<0.05). Because RAG^{-/-} mice are highly susceptible to T. gondii infection similar studies with T. gondii pre-treated mice could not be performed. These results suggest that T and B cells are not required for STAg-mediated protection; however they may be important for the STAg-mediated reduction in viral titers.

The present studies demonstrate that mice previously infected with T. gondii are protected from severe influenza virus infection. Further, treatment with a T. gondii protein extract 48 hours post-viral infection is protective. The studies suggest that T. gondii and STAg may trigger innate immune or inflammatory responses leading to enhanced adaptive responses. This is evident by the early increase in type 1 IFN levels followed by an increased and prolonged elevation in type 2 IFN levels. In the virus alone mice, there is a rapid “collapse” of type 2 IFN levels by 7 dpi likely correlating with the lack of adaptive immune response that is a hallmark of the severe H5N1 influenza virus infections. Lymphopenia is pronounced in humans and animals with fatal H5N1 influenza virus infection. The mechanism remains unknown.

The innate responses alone may play an important role in facilitating viral clearance. Several studies demonstrated the importance of neutrophils in controlling early viral replication during severe influenza virus infections (Tumpey et al., 2005; Perrone et al., 2005). However, the studies in RAG^{-/-} mice suggest that T and/or B cells are also involved in viral clearance in the STAg model. Although it would be easy to speculate that the T. gondii/STAg-mediated protection is solely driven by a decrease in viral replication, 80 to 100% of the N28E2 infected mice and 50% of the STAg-treated RAG^{-/-} mice survived infection with no-to-little decrease in viral titers. This was also observed in mice infected with lower concentrations of WT T. gondii.

The amount of T. gondii cysts present in the brains of mice inoculated with T. gondii was quantitated at 3 and 7 days post infection with avian influenza. T. gondii cysts counts were quantitated from the brains of the mice as described in Mordue et al. (2007). As can be seen in FIG. 5, mice pre-inoculated with the mutant T. gondii strains 73F9 and N28E2 one month prior to infection with avian influenza had significantly fewer T. gondii brain cysts at days 0, 3, and 7 post-infection, as compared to mice pre-inoculated with wild-type T. gondii. No cysts were detected in the brains of mice pre-inoculated with wild-type T. gondii or the mutant 73F9 and N28E2 strains at 22 days post-infection. This suggests that pre-inoculation of mice exposed to avian influenza with wild-type or the mutant T. gondii strains 73F9 and N28E2 may not result in latent T. gondii infection.

The mechanism by which pre-inoculation with T. gondii confers protection against avian influenza was evaluated. Flow cytometry on lung homogenates from the mice in FIG. 6 was performed as described above to determine the presence of neutrophils, antigen presenting cells (APC), CD4⁺ T cells, and CD8⁺ T cells. The markers for the antigen presenting cells were: live staining (violet fixable stain, available from Invitrogen), F4/80 (positive, clone BM8-FITC), Lly-6G (negative, clone RB6-8C5-PE). The markers for the neutrophils were: live staining (violet fixable stain, available from Invitrogen), Ly-6G (positive, clone Rb6-8C5-PE), F4/80 (Negative, clone BM8-FITC). The markers for the CD4⁺ T cells were: live staining (violet fixable stain, available from Invitrogen), CD3 (positive, clone 17A2, Alexa Fluor 647), NK (negative, clone DX5-FITC), CD4 (positive, clone GK1.5-PE), CD8 (negative, clone 53-6.7-APC-eFluor 780). The markers for the CD8⁺ T cells were: live staining (violet fixable stain, available from Invitrogen), CD3 (positive, clone 17A2, Alexa Fluor 647), NK (negative, DX5-FITC), CD8 (positive, clone 53-6.7-APC-eFluor 780), CD4 (negative, clone GK1.5-PE).

FIG. 6A shows the flow cytometry results for day 0 for mice pre-inoculated with either 5000 wild-type T. gondii or pre-inoculated with 5000 or 500 parasites from the mutant T. gondii strains N28E2 or 73F9 one month prior to infection with avian influenza, as compared to an age-matched control group of mice that were not pre-inoculated with T. gondii and were not infected with influenza virus.

FIG. 6B shows the flow cytometry results for day 3 post-infection for mice pre-inoculated with either 5000 wild-type T. gondii or pre-inoculated with 5000 or 500 parasites from the mutant T. gondii strains N28E2 or 73F9 one month prior to infection with avian influenza, as compared to an age-matched control group of mice that were not pre-inoculated with T. gondii but were infected with influenza at day 0 (virus alone). As can be seen from these results, mice pre-inoculated with 5000 parasites from the mutant T. gondii strain 73F9 one month prior to infection with avian influenza had significantly more antigen presenting cells and more neutrophils as compared to the control.

FIG. 6C shows the flow cytometry results for day 7 post-infection for mice pre-inoculated with either 5000 wild-type T. gondii or pre-inoculated with 5000 or 500 parasites from the mutant T. gondii strains N28E2 or 73F9 one month prior to infection with avian influenza, as compared to an age-matched control group of mice that were not pre-inoculated with T. gondii but were infected with influenza at day 0 (virus alone). As can be seen from these results, the amount of CD8+ T cells and the amount of neutrophils at day 7 post-infection was higher for mice pre-inoculated with 5000 wild-type T. gondii or 5000 of the mutant N28E2 strain of T. gondii or 500 of the mutant 73F9 strain of T. gondii, as compared to the control.

Thus, T. gondii may protect during influenza infection by mechanisms dependent and independent of lowered viral titers. T. gondii and STAg will be important tools to understand the immune responses necessary for protection against severe influenza virus infections and may lead to new therapies that specifically upregulate these responses providing protection even after infection.

### SUMMARY

In these studies, it was shown that mice chronically infected with the protistian parasite T. gondii survive lethal influenza infection. T. gondii induced protection is likely not dependent on lowering viral titers, highlighting that the health impact of influenza may not necessarily correlate with viral load. Because a non-infectious fraction of soluble tachyzoite antigens (STAg) elicits an immune response similar to live T. gondii, STAg was tested as a post-infection treatment for influenza virus. STAg administration after lethal influenza infection enhanced survival, lowered viral titers, and reduced clinical disease. Examination of STAg treatment in mice without B and T lymphocytes showed that while an adaptive immune response is not necessary for protection, it is necessary for viral clearance. These studies demonstrate that proteins from one microbe can be used to enhance the immune response to a second (heterologous) microbe. The generation of protection without reduction in viral titers cautions that while new treatments protect against disease, they may simultaneously generate asymptomatic carriers.

### Example II

### Use of T. gondii or Lipoxygenase Preparations to Inhibit or Treat Other Microbial Infections

### Results

The ability of STAg-containing preparations to protect mice infected with the bacteria Listeria monocytogenes was evaluated. 4 to 6 week old C57BL/6 (Jackson Laboratory, Bar Harbor, Me.) mice were intravenously (i.v.) administered 200 μL per mouse PBS, 200 μL per mouse STAg (prepared as described above) with the T. gondii strain N28E2 or 10⁴ units of soybean lipoxygenase (Sigma) per mouse. Four mice were used for each treatment group. 24 hours after pre-treatment with the PBS, lipoxygenase or STAg treated mice were infected intragastrically with L. monocytogenes and 3 days after infection, the liver and spleen of the mice were tested for the presence of L. monocytogenes. The limits of detection (CFU) were approximately 2.5 for the liver and 3.2 for the spleen. The results are shown in FIG. 7. Only one of the four STAg treated mice had any detectable bacteria, and the detectable bacteria were in the spleen of that mouse, demonstrating that STAg is effective at protecting mice against infection with L. monocytogenes. STAg was equally effective at protecting mice against infection with L. monocytogenes when administered by intraperitoneal injection (data not shown).

The ability of STAg-containing preparations to treat mice infected with the bacteria Listeria monocytogenes was evaluated. 4 to 6 week old C57BL/6 (Jackson Laboratory, Bar Harbor, Me.) mice were infected with L. monocytogenes. Twenty-four hours after infection, mice were intravenously (i.v.) administered either 200 μL per PBS, 200 μL per mouse STAg (prepared as described above with the T. gondii strain N28E2), or 200 μL of STAg (prepared with the T. gondii strain N28E2) digested with 100 μg/mL of proteinase K. Eight mice were used for each treatment group. Mice were sacrificed three days after infection with L. monocytogenes and their spleens were harvested, ground, diluted, and plated. The number of colony forming units (CFU) was measured. The results are shown in FIG. 8.

As can be seen from FIG. 8, STAg is an effective treatment for L. monocytogenes infections when administered 24 hours after bacterial infection. Additionally, proteinase K treatment of STAg ablates its protective effects against L. monocytogenes infection, indicating that the active component in STAg is likely a protein.

One third of the world's most populous countries are at risk for malaria. Each year 350-500 million cases of malaria occur worldwide, and over one million people die, most of them young children in Sub-Saharan Africa. Cerebral malaria is a fatal complication of Plasmodium falciparum infection that claims hundreds of thousands of lives a year. Despite the vast literature on the pathogenesis of cerebral malaria, the mechanisms leading to severe brain pathology and death remain largely unknown. T cells and pro-inflammatory cytokines contribute to the brain inflammation, but how they mediate pathology is unclear. Consequently, no specific treatment for cerebral malaria exists. While there is no perfect animal model for human P. falciparum induced cerebral malaria (P. falciparum does not infect mice), the rodent-specific species P. berghei is widely used as it induces lethal brain inflammation and vascular damage similar to P. falciparum in humans. In susceptible mouse strains, such as C57BL/6, a high inoculum will paralyze mice usually between six to eight days post infection, and death follows shortly after the onset of paralysis.

The ability of STAg-containing preparations to treat mice infected with Plasmodium berghei was evaluated. P. berghei is a rodent-specific species of Plasmodium that induces lethal brain inflammation and vascular damage similar to P. falciparum in humans, and is widely used as a mouse model of cerebral malaria. In this experiment, 4 to 6 week old C57BL/6 (Jackson Laboratory, Bar Harbor, Me.) mice were infected with 10⁶ red blood cells parasitized with P. berghei. At 48 and 96 hours post infection (2 treatments, i.e., double dose) mice were intravenously administered either 200 μL per mouse of PBS or 200 μL per mouse of STAg (prepared as described above with the T. gondii strain N28E2). Three mice were used for each treatment group. The mice were monitored for signs of infection every 12 hours starting at 5 days post-infection. Mice treated with PBS only were sacrificed at days 7 and 7.5 due to severe paralysis and convulsions. One of the three STAg-treated mice was sacrificed at day 10 when it developed paralysis. The other two STAg treated mice were sacrificed at day 20 (last day of the experiment), but were not symptomatic.

As can be seen from FIG. 9, all of the mice treated with PBS alone developed severe paralysis and had to be sacrificed. In contrast, two of the three mice treated with STAg were asymptomatic up to 20 days post-infection when the experiment was ended, indicating that STAg may be effective at treating mice infected with P. berghei.

### Example III

### Use of Lipoxygenase Preparations to Inhibit or Treat Microbial Infections

Lipoxygenase isolated from cells, e.g., from non-recombinant cellular sources including cells infected with Toxoplasma and plant cells, e.g., soybean cells, or from recombinant sources, for instance, cell expressing a recombinant nucleic acid encoding a lipoxygenase such as one having SEQ ID NO:1, may be employed in compositions of the invention. In one embodiment, a composition of the invention has an amount of isolated lipoxygenase effective to prevent microbial, e.g., viral, bacterial, fungal or parasite, infection. In another embodiment, a composition of the invention has an amount of isolated lipoxygenase effective to inhibit microbial, e.g., viral, bacterial, fungal or parasite, infection or replication. In yet another embodiment, a composition of the invention has an amount of isolated lipoxygenase effective to treat microbial, e.g., viral, bacterial, fungal or parasite, infection.

For example, a mammal such as a human is administered, for instance, intravenously, an effective amount of eukaryotic lipoxygenase (e.g., a plant lipoxygenase such as soybean, potato and eggplant, or a Toxoplasma lipoxygenase), prior to or after microbial infection including but not limited to influenza virus, Listeria or Plasmodium infection. In one embodiment, a mammal, e.g., a mouse, ferret or human, is administered a soluble extract of Toxoplasma or isolated lipoxygenase, e.g., intravenously or intranasally, about 2 to 4 days after a suspected exposure to influenza virus. For example, a mouse or ferret is contacted, for instance, intranasally, with an infectious dose of influenza virus and about 2 days later the animal is administered a soluble extract of Toxoplasma or isolated lipoxygenase in an amount that inhibits or treats the viral infection. In one embodiment, a mammal, e.g., a mouse, ferret or human, is administered a soluble extract of Toxoplasma or isolated lipoxygenase, e.g., intravenously or intranasally, before, such as about 1 to 4 months before a possible exposure to influenza virus. For example, a mouse or ferret is contacted, for instance, intranasally, with an amount of a soluble extract of Toxoplasma or isolated lipoxygenase that prevents or inhibits viral infection or replication, such as when the animal is challenged with an infectious dose of influenza virus 1 to 4 months after administration of the soluble extract of Toxoplasma or isolated lipoxygenase.

### Example IV

A protocol to induce T. gondii parasites to egress host cells within minutes was employed to obtain host-cell free T. gondii preparations. Infected cells were treated with a calcium ionophore (A23187) which allows for easy and rapid collection of parasites. STAg prepared from egressed parasites was just as effective against influenza A virus, Listeria and Plasmodium infection as STAg produced from syringe lysed parasites. Moreover, STAg preparation time was reduced from several hours to about 10 minutes.

STAg preparations from a T. gondii knock-out strain of SEQ ID NO:1 (putative lipoxygenase) had a similar activity to STAg preparations from wild-type T. gondii.

### REFERENCES

Bermudez et al., Infect Immun., 61: 4126 (1993).
Charest et al., J. Immunol., 165:2084 (2000).
Frankel et al., Proc. Natl. Acad. Sci., 104:10181 (2007).
Gentry and Remington, J. Infect. Dis., 123:22 (1971).
Hunter et al., Infect Immun., 61:4038 (1993).
Jones et al., J. Virol., 80:11960 (2006).
La Gruta et al., Immunol. Cell. Biol., 85:85 (2007).
Mahmoud et al., Nature, 263:56 (1976).
Maines et al., Immunol. Rev., 225:68 (2008).
Mordue et al., Mol. Micro., Vol. 63:482 (2007).
Morton et al., ILAR, 41:80 (2000).
Perrone et al., PLoS Pathogens, 4:e1000115, 1 (2008).
Price et al., J. Virol., 74 :3996 (2000).
Reagan-Shaw et al., FASEB, 22:659 (2008).
Reed and Muench, Am. J. Hyg., 27:493 (1938).
Remington and Merigan, Proc. Soc. Exp. Biol. Med., 131:1184 (1969).
Ruskin and Remington, Science, 160:72 (1968).
Schoenborn & Wilson, Adv. Immunol., 96:41 (2007).
Suzuki et al., Science, 240:516 (1988).
Tumpey et al., J. Virol., 74:6105 (2000).
Tumpey et al., J. Virol., 79:14933 (2005).
Ware & Kasper, L. H., Infect. Immun., 55:78 (1987).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A composition comprising an amount of i) a soluble extract of a live attenuated or avirulent Toxoplasma gondii strain, ii) a soluble extract of a wild-type T. gondii strain or iii) a live attenuated or an avirulent Toxoplasma gondii strain, effective to inhibit or prevent heterologous microbial pathogen infection or replication, or one or more symptoms or manifestations of the microbial pathogen infection or replication.

2. The composition of claim 1 comprising T. gondii strain N28E2 or an extract thereof.

3. The composition of claim 1 comprising T. gondii strain 73F9 or 41E2 or an extract thereof.

4. The composition of claim 1 further comprising a pharmaceutically acceptable carrier.

5. The composition of claim 1 which comprises from about 20 mg to about 500 mg of extracted soluble T. gondii protein.

6. A composition comprising an amount of one or more isolated T. gondii proteins effective to inhibit or prevent heterologous microbial pathogen infection or replication, or one or more symptoms or manifestations of the microbial pathogen infection or replication, wherein one of the proteins is T. gondii lipoxygenase, and optionally comprising one or more immunogens, one or more stabilizers, one or more preservatives, or combinations thereof.

7. (canceled)

8. A method to prevent or inhibit microbial pathogen infection or replication in a mammal, comprising administering to the mammal a composition comprising an amount of a live attenuated or avirulent T. gondii strain or a soluble extract thereof, a soluble extract of a wild-type T. gondii strain, a recombinant T. gondii strain that expresses one or more immunogens of a heterologous microbial pathogen or a soluble extract thereof, or isolated lipoxygenase, effective to prevent or inhibit a heterologous microbial pathogen infection, wherein the composition optionally comprises a pharmaceutically acceptable carrier.

9. The method of claim 8 wherein the heterologous microbial pathogen is a virus, bacterium or fungus.

10. The method of claim 8 wherein the virus is influenza virus.

11. The method of claim 8 wherein the heterologous microbial pathogen is a parasite.

12-13. (canceled)

14. The method of claim 8 wherein the composition comprises a live avirulent T. gondii strain or a soluble extract thereof.

15. The method of claim 8 wherein the attenuated or avirulent strain is 73F9 or N28E2.

16. (canceled)

17. The method of claim 8 wherein the composition is administered after the mammal is exposed to the heterologous microbial pathogen.

18. The method of claim 8 wherein the composition is administered to the mammal prior to exposure to the heterologous microbial pathogen.

19. The method of claim 8 wherein the composition is parenterally, intranasally, intramuscularly or orally administered.

20-22. (canceled)

23. The method of claim 8 any one of claims 8 to 22 wherein the mammal is a human.

24. (canceled)

25. A method to augment an immune response in a mammal, comprising administering to a mammal a composition comprising an immunogen for a microbial pathogen and an amount of a live attenuated or avirulent T. gondii strain or a soluble extract thereof, a soluble extract of a wild-type T. gondii strain, a recombinant T. gondii strain that expresses one or more antigens of the microbial pathogen or a soluble extract thereof, or isolated lipoxygenase, effective to augment the immune response in the mammal to the immunogen.

26. A composition comprising: an amount of a live attenuated or avirulent T. gondii strain or a soluble extract thereof, a soluble extract of a wild-type T. gondii strain, a recombinant T. gondii strain that expresses one or more antigens of the microbial pathogen or a soluble extract thereof, or isolated lipoxygenase, effective to augment an immune response to a heterologous immunogen.

27. The composition of claim 26 wherein the heterologous immunogen includes recombinant influenza virus HA.
